SEQUENCE LISTING (10 sequences)

SEQ ID NO: 1 - protein, 1202 aa, mus musculus
MSFFNFRKIFKLGSEKKKKQYEHVKRDLNPEEFWEIIGELGDGAFGKVYKAQNKETNVLAAAKVIDTKSEEELEDYMVEIDILASCDHPNIVKLLDAFYYENNLWILIEFCAGGAVDAVMLELERPLTESQIQVVCKQTLEALNYLHDNKIIHRDLKAGNILFTLDGDIKLADFGVSAKNTRTIQRRDSFIGTPYWMAPEVVMCETSKDRPYDYKADVWSLGITLIEMAEIEPPHHELNPMRVLLKIAKSEPPTLAQPSKWSSNFKDFLRKCLEKNVDARWTTSQLLQHPFVTVDSNKPVRELIAEAKAEVTEEVEDGKEEDEEEEAENALPIPANKRASSDLSIASSEEDKLSQNACILESVSERTEQSTSEDKFSNKILNEKPTTDGPEKAVDEHASDVNLETGAELNDQTVGIHENGREKKRPKLENLPDTQDQQTVDVNSVSEENENNRVTLETNTDCLKPEEDRNKENQETLESKLIQSEEINDTHIQTMDLVSQETGEKEADFQAVDNEVGLTKEETQEKLGKDGTAQKVITSDRSSEVGTDEALDDTQKAAELSKAAQSGEGDEALAPTQTLAEKPTEGPEAGGAEEEPPGGERVEDKQPEQQPAVCEAEGQLTSTSETTRATLEQPETDEVEQVSESNSIEELERLVVTGAEARALGSEGEAAATEVDLERKENAQKVPVKAESQAPAASQPSEPHPVLIPSININSETTENKEEMGALPKPETILPPEPEHEKGNDTDSGTGSTVENSSGDLNLSISSFLSKAKDSGSVSLQETRRQKKTLKKTRKFIVDGVEVSVTTSKIVTDSDSKTEELRFLRRQELRELRLLQKEEQRAQQQLNGKLQQQREQIFRRFEQEMLSKKRQYDQEIENLEKQQKQTIERLEQEHTNRLRDEAKRIKGEQEKELSKFQNVLKNRKKEEQEFVQKQQQELDGSLKKIIQQQKAELANIERECLNNKQQLMRAREAAIWELEERHLQEKHQLLKQQLKDQYFMQRHQLLKRHEKETEQMQRYNQRLIEELKNRQTQERARLPKIQRSEAKTRMAMFKKSLRINSTATPDQDREKIKQFAAQEEKRQKNERMAQHQKHESQMRDLQLQCEANVRELHQLQNEKCHLLVEHETQKLKELDEEHSQELKEWREKLRPRKKTLEEEFARKLQEQEVFFKMTGESECLNPSAQSRISKFYPIPTLHSTGS

SEQ ID NO: 2 - protein, 1202 aa, Artificial Sequence
  K63R SLK Inhibitor SNP of mus musculus
MSFFNFRKIFKLGSEKKKKQYEHVKRDLNPEEFWEIIGELGDGAFGKVYKAQNKETNVLAAARVIDTKSEEELEDYMVEIDILASCDHPNIVKLLDAFYYENNLWILIEFCAGGAVDAVMLELERPLTESQIQVVCKQTLEALNYLHDNKIIHRDLKAGNILFTLDGDIKLADFGVSAKNTRTIQRRDSFIGTPYWMAPEVVMCETSKDRPYDYKADVWSLGITLIEMAEIEPPHHELNPMRVLLKIAKSEPPTLAQPSKWSSNFKDFLRKCLEKNVDARWTTSQLLQHPFVTVDSNKPVRELIAEAKAEVTEEVEDGKEEDEEEEAENALPIPANKRASSDLSIASSEEDKLSQNACILESVSERTEQSTSEDKFSNKILNEKPTTDGPEKAVDEHASDVNLETGAELNDQTVGIHENGREKKRPKLENLPDTQDQQTVDVNSVSEENENNRVTLETNTDCLKPEEDRNKENQETLESKLIQSEEINDTHIQTMDLVSQETGEKEADFQAVDNEVGLTKEETQEKLGKDGTAQKVITSDRSSEVGTDEALDDTQKAAELSKAAQSGEGDEALAPTQTLAEKPTEGPEAGGAEEEPPGGERVEDKQPEQQPAVCEAEGQLTSTSETTRATLEQPETDEVEQVSESNSIEELERLVVTGAEARALGSEGEAAATEVDLERKENAQKVPVKAESQAPAASQPSEPHPVLIPSININSETTENKEEMGALPKPETILPPEPEHEKGNDTDSGTGSTVENSSGDLNLSISSFLSKAKDSGSVSLQETRRQKKTLKKTRKFIVDGVEVSVTTSKIVTDSDSKTEELRFLRRQELRELRLLQKEEQRAQQQLNGKLQQQREQIFRRFEQEMLSKKRQYDQEIENLEKQQKQTIERLEQEHTNRLRDEAKRIKGEQEKELSKFQNVLKNRKKEEQEFVQKQQQELDGSLKKIIQQQKAELANIERECLNNKQQLMRAREAAIWELEERHLQEKHQLLKQQLKDQYFMQRHQLLKRHEKETEQMQRYNQRLIEELKNRQTQERARLPKIQRSEAKTRMAMFKKSLRINSTATPDQDREKIKQFAAQEEKRQKNERMAQHQKHESQMRDLQLQCEANVRELHQLQNEKCHLLVEHETQKLKELDEEHSQELKEWREKLRPRKKTLEEEFARKLQEQEVFFKMTGESECLNPSAQSRISKFYPIPTLHSTGS

SEQ ID NO: 3 - DNA, 19 bp, Artificial Sequence
  murine ALK target sequence
ggttgagattgacatatta

SEQ ID NO: 4 - DNA, 19 bp, Artificial Sequence
  murine ALK target sequence
gataatttatggatgtgac

SEQ ID NO: 5 - DNA, 16 bp, Artificial Sequence
  murine ALK target sequence
agcgacaaacacacaa

SEQ ID NO: 6 - DNA, 12 bp, Artificial Sequence
  murine SiRNA Knockdown of SLK
gggagagacaaa

SEQ ID NO: 7 - DNA, 17 bp, Artificial Sequence
  murine SiRNA Knockdown of Ldb2
acaagcagcacgccaaa

SEQ ID NO: 8 - DNA, 15 bp, Artificial Sequence
  murine SiRNA Knockdown of Ldb1
gaacagcccgccaca

SEQ ID NO: 9 - DNA, 32 bp, Artificial Sequence
  murine PCR primer amplfying Ldb2 LBD
ggggggatccagctgcaaacctgagtctgtcc

SEQ ID NO: 10 - DNA, 34 bp, Artificial Sequence
  murine PCR primer amplfying Ldb2 LBD
gggggaattcacgggcctattgacagtggattct

(19) United States
(12) Patent Application Publication
(10) Pub. No.: US 2009/0081195 A1
(43) Pub. Date: Mar. 26, 2009

(54) Inhibitors of Ste20-like Kinase (SLK) and Methods of Modulating Cell Cycle Progression and Cell Motility

(75) Inventor: Luc A. Sabourin, Gatineau (CA)

Correspondence Address: SUZANNAH K. SUNDBY;SMITH, GAMBRELL & RUSSEL, LLP, 1130 Connecticut Avenue, NW, Suite 1130, WASHINGTON, DC, 20036, US

(73) Assignee: OTTAWA HEALTH RESEARCH INSTITUTE, Ottawa (CA)

(21) Appl. No.: 12/067,844

(22) PCT Filed: Sep. 29, 2006
(86) PCT No.: PCT/CA2006/001613

§ 371 Date: Jul. 31, 2008

Related U.S. Application Data
(60) Provisional application No. 60/722,080, filed on Sep. 30, 2005.

(51) Int. Cl. A61K 39/395; A61K 35/76; C12N 5/00; C12Q 1/48; A61P 35/00

(52) U.S. Cl. 424/130.1; 424/93.6; 435/325; 435/375; 435/15

(57) ABSTRACT

The invention provides inhibitors of Ste20-like kinase (SLK inhibitors). The inhibitors may be employed to modulate proliferation of cells, including tumor and cancer cells. The inhibitors also may be employed to inhibit motility or migration of cells, including cancer and tumor cells.

The present invention relates to kinase inhibitors. More specifically, the present invention relates to inhibitors of Ste20-like kinase (SLK) and methods of modulating cell cycle progression and motility of cells using such inhibitors.

### BACKGROUND OF THE INVENTION

Cell cycle progression is monitored through kinase mediated signal transduction and the binding of various cyclin proteins to their respective cyclin-dependent kinases (Cdks; (2,3)). The activity of a cyclin/Cdk complex is regulated by cycles of expression and destruction of the cyclin subunit (reviewed in (4)).

G1 progression is regulated, in part, by cyclins D and E and their respective cyclin dependent kinases in a complex pathway that results in the retinoblastoma (Rb) protein phosphorylation and consequently the production of cyclin A leading to S phase entry (Reviewed in (5)). Cyclin B synthesis initiates at the end of S phase (6,7) and forms a complex with p34cdc2/cdk1. This complex has been termed MPF (maturation promoting factor or mitosis promoting factor) and is required for mitotic entry (reviewed in (8)). During interphase, cytosolic MPF is kept inactive by inhibitory phosphorylation of cdc2 on threonine 14 (Thr14) and tyrosine 15 (Tyr15) by Myt1 and Wee1, respectively (9-11). Activation of this complex is triggered by the Cdc25C phosphatase through cdc2 dephosphorylation of Thr14 and Tyr 15 (12-14). Following dephosphorylation of these residues, MPF is believed to phosphorylate and further activate Cdc25C resulting in full activation of MPF through an autocatalytic feedback loop (15,16). This results in the translocation of MPF from the cytoplasm to the nucleus at the beginning of mitosis (17) where it phosphorylates histone H1 (18) and induces changes in the microtubule network (19) and actin filaments (20).

In Xenopus, polo like kinase (Plx1) has been shown to phosphorylate and activate Cdc25 (21) and polo like kinase (xPlkk1) has been shown to be a direct activator of Plx1 (22). However, this may be an organism specific phenomenon since depletion of mammalian polo like kinase (Plk1) results in elevated activity of Cdc2 (23) suggesting a role for Plk in mitotic progression rather than mitotic entry. To date, a bona fide upstream activator of mammalian Cdc25C has not been identified.

Chromosome condensation is accompanied by the hyperphosphorylation of histone H1 (24) and phosphorylation of H3 on serine 10 (Ser10) (25,26). Microtubules then organize into a bipolar spindle and attach to the kinetochores of each sister chromosome. Chromosome attachment prior to segregation is monitored by the spindle check point protein MAD2 (mitotic arrest deficient) which binds kinetochores lacking microtubule attachment generating a “wait-anaphase” signal (Reviewed in (27)). Following the attachment of the last unattached kinetochore, the “wait anaphase” signal is silenced and the anaphase-promoting complex (APC), in association with Cdc20 initiates chromosome segregation (Reviewed in (28)) and culminates into cytokinesis (Reviewed in (29)).

The murine Ste20-like kinase (SLK) is a 220 kDa serine/threonine kinase that was first demonstrated to induce actin remodeling and apoptosis in a wide range of cell lines (30,31). The amino terminal kinase domain of SLK is closely related to that of lymphocyte oriented kinase (LOK) and xPlkk1. In addition, SLK bears a central microtubule and nuclear associated protein (M-NAP) domain, and a carboxyl AT1-46 homology (termed ATH) domain that is also found in LOK and xPlkk1 (30,31). The function of both the M-NAP and AT1-46 domains has yet to be elucidated. SLK is expressed early in development, preferentially in neuronal and myogenic lineages, and ubiquitously in adult tissue (32). It has been shown to co-localize with adhesion markers during cell spreading, is intimately linked to the microtubule network (1), has been shown to phosphorylate and activate Plk (33), and is required for fusion of C2C12 myoblasts (43). SLK has also been shown to regulate cell cycle progression (44).

Several LIM domain-containing proteins have roles in cell migration. LIM kinases are required for actin dynamics during directional migration (45) and paxillin modulates focal adhesion turnover, a process required for cell migration (46). Ldb1 (CLIM2, NL1) and the highly related paralog Ldb2 (CLIM1) (hereafter referred to Ldb2 and Ldb1) are LIM domain binding transcription co-factors required for the function of LIM homeodomain transactivators (47-49). Appropriate interactions of these factors are important for normal neuronal sub-type identity and development (50,51).

Here we show that SLK is implicated in cell proliferation, motility and migration and further, that the Ldb1/2 co-activators interact with SLK in vitro and in vivo. We also show that Ldb1 and 2 function to inhibit SLK activity in vitro. Furthermore we establish that Ldb2 but not Ldb1 can bind directly to α-tubulin, providing a link between SLK and the microtubule network. Finally, Cre-mediated excision of Ldb1 or treatment of fibroblasts with siRNA for Ldb1 and 2 results in a 4-fold increase in migration rate in a Boyden chamber haptotaxis assay thus ascribing a novel extranuclear function to Ldb1 and 2 as regulators of SLK and cell migration.

WO 00/49139 discloses a caspase activated protein kinase called SMAK which is identical in amino acid sequence to murine Ste20-like kinase. The reference discloses that SMAK activates two distinct signalling pathways that are involved in mediating apoptosis. Further, the reference notes that it may not be desirable to inhibit the expression of the SMAK protein as the protein appears to be associated with apoptosis and may play a role in preventing neoplasia development.

There is a need in the art to identify compounds that inhibit the proliferation of cells. Further, there is a need in the art for compounds that inhibit the proliferation of cancers or tumor cells. There is also a need in the art to identify compounds that inhibit the motility or migration of cells. There is a further need in the art for compounds that inhibit cancer or tumor cells from metastasizing.

### SUMMARY OF THE INVENTION

The present invention relates to kinase inhibitors. More specifically, the present invention relates to inhibitors of Ste20-like kinase (SLK) and methods of modulating cell cycle progression and motility of cells using such inhibitors.

According to the present invention there is provided a method of inhibiting proliferation, motility or both proliferation and motility of a cell, for example, but not limited to a cancer or tumor cell in a subject, the method comprising administering an SLK inhibitor to said subject. The subject may be an animal subject, preferably a mammalian subject, more preferably a human subject.

The present invention also provides a method as defined above, wherein the SLK inhibitor is an antisense nucleic acid, a short interfering RNA (siRNA), a catalytically inactive SLK or a nucleic acid encoding a catalytically inactive or kinase-dead SLK. The SLK inhibitor may also comprise a fragment of SLK that is catalytically inactive or kinase-dead.

The present invention also provides a method as defined above wherein the SLK inhibitor is an antibody or fragment thereof which is capable of binding SLK.

The present invention also provides a method as defined above, wherein the SLK inhibitor is a binding partner of SLK, a variant thereof, or a fragment of a binding partner of SLK that is capable of binding to SLK and inhibiting the activity of SLK. In a preferred embodiment, the SLK inhibitor is a heterologous SLK inhibitor meaning an inhibitor that does not exist naturally in a cell.

The present invention also provides for the use of an SLK inhibitor to treat a cancer or tumour in a subject. Further the SLK inhibitor may be employed for the production of a medicament to treat a cancer or tumour in a subject.

The present invention also provides a cell comprising an SLK inhibitor. In a preferred embodiment the cell is a non-cultured cell, preferably a non-cultured cancer or a tumour cell. In still a further embodiment, the cell is a HER2+ carcinoma or sarcinoma.

The present invention also contemplates SLK inhibitors that can be used in the methods as described above.

Also contemplated by the present invention is an in vitro method of inhibiting the proliferation, motility or both the proliferation and motility of a cell comprising administering an SLK inhibitor to said cell. In a preferred embodiment, which is not meant to be limiting in any manner, the cell is a cancer or tumor cell. In a more preferred embodiment the cell is a human cell.

Also contemplated is an in vitro method as defined above, wherein the SLK inhibitor is encoded by a virus, for example, but not limited to a lentivirus, adenovirus or a retrovirus.

The present invention also contemplates an in vitro method as defined above wherein the SLK inhibitor comprises a catalytically inactive SLK or a nucleic acid encoding a catalytically inactive SLK kinase.

Further, the present invention contemplates an in-vitro method as defined above wherein the SLK inhibitor comprises a fragment of SLK. Also provided are in vitro methods wherein the SLK inhibitor is an antisense nucleic acid or a short interfering RNA (siRNA), an antibody or fragment thereof which is capable of binding SLK, a binding partner of SLK, a variant of a naturally occurring binding partner, or a fragment of a binding partner of SLK.

The present invention also provides a method of screening a compound to determine if said compound is effective as an anticancer agent, the method comprising,
a) selecting a first group of cells and a second group of cells,
b) treating the first group of cells with the compound;
c) measuring SLK kinase activity, proliferation, motility, migration or any combination thereof of said first group of cells relative to the second group of cells, wherein a measurable decrease in SLK kinase activity, proliferation, motility, migration or any combination thereof in said first group of cells relative to said second group of cells indicates that said compound is effective as an anticancer agent.

It is generally preferred that the second group of cells is treated with an appropriate control under conditions substantially similar to the first group of cells. Further, in an alternate embodiment, which is not meant to be limiting in any manner, the first group of cells are cancer or tumor cells and the second group of cells are non-cancerous normal cells of the same cell type. In a further embodiment, the non-cancerous normal cells and the cancer or tumor cells are from the same initial cell line. In such cases, the first group of cells may be made cancerous by any means known in the art. In still a further embodiment, the non-cancerous normal cells and the cancer or tumor cells may be obtained from a subject, for example, a human subject.

This summary of the invention does not necessarily describe all necessary features of the invention.

### BRIEF DESCRIPTION OF THE FIGURES

FIG. 1 shows results suggesting SLK localizes to the mitotic spindle. Double labeling and confocal analysis shows that SLK (A) also co-localizes with tubulin (B) at the mitotic spindle. Chromosomes were visualized with DAPI (C). An overlay of these stains displays that SLK localizes to the mitotic spindle (D).

FIG. 2 shows results suggesting SLK activity increases during G2/M and is required for proliferation. C3H10T1/2 cells were synchronized to quiescence by 48 hours of serum deprivation and released by the addition of 20% serum. Cells were then collected at different times and monitored by flow cytometric analysis (A) and SLK activity (B). SLK expression does not change while its activity increases at G2/M. Probing of the immunoprecipitates with an anti-SLK confirmed that equivalent amounts of SLK were present in the kinase assays. (C) PGK-Puro and PGK-Puro-AS-SLK (antisense SLK) were stably transfected into C3H10T1/2 cells by selection for puromycin resistance over 14 days. Stable clones were then visualized by staining with CYTO-QUIK. Cells transfected with an antisense SLK vector display a marked decrease in stable clone number, suggesting a proliferative block. (D) Cultures infected with an adenovirus bearing a kinase inactive version of SLK (KΔC) were collected over time and viable cells were counted by trypan blue exclusion. Cells infected with KΔC displayed a marked reduction in proliferation.

FIG. 3 shows results suggesting expression of kinase inactive SLK results in a G2/M block. C3H10T1/2 cells infected with adenovirus carrying LacZ or HA-KΔC were synchronized to quiescence by serum deprivation and then released by the addition of 20% serum. Cells were then monitored by flow cytometric DNA content analysis. After 32 hours of serum stimulation, HA-KΔC expressing cells show a delay in G2/M transit time when compared to control infected cultures (A and B). Supporting this, BrdU labeling of exponentially growing cultures and DNA content monitoring of BrdU positive cells shows that HA-KΔC infected cells proceed through G2/M with delayed kinetics (C). A representative of four independent experiments is shown.

FIG. 4 shows results suggesting kinase inactive SLK alters cyclin A expression pattern and p34/Cdc2 activation. (A) C3H10T1/2 cells brought to quiescence by 48 hours of serum deprivation and infected with KΔC or lacZ viruses were monitored for cell cycle regulating proteins after serum stimulation by Western blot analysis. The expression patterns of cyclins D, E, B, and p34/cdc2 were not found to differ markedly between LacZ or KΔC-infected cultures. However, the levels of cyclin A protein in KΔCexpressing cells were found to remain elevated (arrowheads), suggesting a G2 block. Infection was confirmed by the HA tag of KΔC and even loading was evaluated by actin levels. (B) Western blot analysis of synchronized fibroblasts transiting through G2/M following infection with HA-KΔC virus or LacZ control and serum stimulation. A marked reduction in Cdc2 activation was observed in KΔC-expressing cultures. The last lane represents a control M phase-synchronized extract.

FIG. 5 shows results suggesting KΔC-expression inhibits histone H3 phopshorylation. Serum starved C3H10T1/2 cells were infected with LacZ (A, B and C) or KΔC (D, E and F) encoding viruses and serum stimulated for 24, 28 or 32 hours. Cells were fixed and stained for anti-HA (D) or β-galactosidase (A) in conjunction with anti-phospho-H3 (pH3; B and E). Nuclei were visualized by DAPI countersatining (C and F). A marked reduction in pH3 staining was observed in KΔC expressing cells. (G) Serum starved C3H10T1/2 cells were stimulated with 20% FCS and double stained for phospho-H3 and HA, or LacZ, at the indicated time points (hours). Double positive HA (or LacZ) and phospho-H3 cells were scored. The results are shown for 3 independent infections where at least 200 nuclei were counted.

FIG. 6 shows results suggesting a role for SLK in G2 progression. Exponentially growing C3H10T1/2 were transfected with the SLK siRNA pool or siCONTROL and analysed by Western blot for SLK expression (A) and by flow cytometry for DNA content (B) 48 hours posttransfection. The samples labeled “control” correspond to cells transfected with the Dharmacon siCONTROL RNA. Identical results were obtained with a SLK “scrambled” siRNA (not shown). A marked downregulation of SLK at 50 nM of siRNA resulted in the accumulation of the cells in the G2/M compartment, further supporting a requirement for SLK for progression through G2. (C) Exponentially growing LLCPK-1 fibroblasts expressing GFP-tubulin were microinjected with an activated form of SLK (HA-tagged; aa 1-373). All (n=25) injected cells (arrowheads), detected by anti-HA staining, displayed ectopic mitotic spindles when expressing activated SLK (panels I and III; merged fluorescence), suggesting that SLK induces mitotic entry. No spindle formation was observed when cells were injected with the kinase dead version (panels II and IV). (D) Xenopus oocytes injected with the same form of activated SLK, re-entered the cell cycle as evidenced by GVBD and the shift in the molecular weight of Plx1, indicative of phosphorylation (pPlx1), suggesting that SLK can activate mitotic entry in Xenopus eggs. As a control, activation of Plx1 by progesterone (P) was used (C=untreated). Expression of kinase dead SLK (mSLK-KD) could not induce oocyte maturation. The GVBD data represent the average of 4 independent experiments. Western blot analysis shows the expression of the Myc-tagged SLK protein.

FIG. 7 shows a nonlimiting depiction of integrin-stimulated signaling events involving FAK. Integrin receptor α/β engagement stimulates FAK autophosphorylation at Tyr-397 and docking of c-src onto the newly generated SH2-binding site. Recruitment of c-src to adhesion sites induces tyrosine phosphorylation of p130Cas inducing the recruitment of Crk and Nck, modulators of Rac and JNKs. Src-mediated phosphorylation of FAK creates a binding site for Grb2 at Tyr-925 and Shc at Tyr-397. Integrin stimulation of Ras through SOS activates the PI 3′-kinase survival pathway and ERK survival. The downregulation of SLK by Src and the potential regulation of MARK3, Rac 1 and Paxillin is illustrated. Drawing adapted from Schlaepfer, et al. (1999). “Signaling through focal adhesion kinase.” Prog Biophys Mol Biol 71(3-4): 435-78, which is herein incorporated by reference.

FIG. 8 shows colocalization of SLK and adhesion components during fibroblast wound healing. Confluent fibroblast monolayers were scratch wounded and allowed to migrate for 4-6h on fibronectin-coated substrates. Cells were then fixed and immunostained for the various proteins. SLK was found to localize at the leading edge with several adhesion markers.

FIG. 9 shows colocalization of SLK and adhesion components during fibroblast wound healing. Confluent fibroblast monolayers were scratch wounded and allowed to migrate for 4-6 h on fibronectin-coated substrates. Cells were then fixed and immunostained for the various proteins. SLK was found to localize at the leading edge with Rac1 and GSK3-β.

FIG. 10 shows results suggesting SLK depletion inhibits haptotaxis on fibronectin. Fibroblasts treated with SLK siRNAs (A) were plated in Boyden Chambers were allowed to migrate for 8 h and then stained with DAPI (B). The underside of the support was quantitated for migratory cells relative to BSA. (C) 1-BSA control; 2-Fibronectin control; 3-siRNA BSA; 4-siRNA fibronectin.

FIG. 11 shows results indicating regulation of cell migration through CLIM2-SLK interaction. (A) Direct binding between SLK C-ter and CLIM2. (B) IP-Western showing in vivo complex between SLK and CLIM2. (C) Co-localization of SLK, CLIM2 and paxillin. (D) IP kinase assays showing increased SLK IP and CLIM1 complex disruption in the absence of CLIM2. (E) Cre-mediated deletion of CLIM2 increases cell motility.

FIG. 12 shows results that dominant negative-SLK (DN-SLK) interferes with cell motility. (A) Expression of SLK in human breast carcinoma lines. (B) HeLa cells expressing virally transduced DN-SLK. (C) Motility is impaired in HeLa cells expressing DN-SLK. (D) Recruitment of SLK into lamellipodia following 1 nm HRG treatment in MCF-7 cells.

FIG. 13 shows activation of SLK by activated ErbB2 and SLK-nm23 interaction. (A) IP kinase assay showing that SLK is activated by activated and complexed by ErbB2. (B) Western analysis of total cell lysates showing expression of exogenous SLK and ErbB2. (C) Inhibition of SLK activity in vitro by nm23. Autophosphorylation for both proteins is shown. (D) Schematic representation of Neu add-back mutants. (D) Pull down assay showing direct binding between SLK and nm23. (E) Induction of adhesion breakdown by expression of a CLIM2 mutant.

FIG. 14 shows results suggesting that Paxillin is phosphorylated by SLK. (A) Schematic of paxillin showing the 5 LD repeats and 4 LIM domains. (B) SLK immunoprecipitates were incubated with GST-paxillin, GST-LD or GST-LIM domains in the presence of 32 Pg-ATP. Paxillin (*) and an LD1-5 N-terminal breakdown product (**) are phosphorylated by SLK.

FIG. 15 shows results indicating that SLK is activated by scratch wounding and is required for cell migration. (a) Confluent MEF3T3 fibroblasts plated on fibronectin (10 μg/ml) and were stimulated to migrate by scratch wounding. SLK kinase activity was assayed in vitro and found to increase over time reaching a maximum activity by 60 minutes following wounding. Total immunoprecipitated SLK levels are shown (lower panel). (b) Knockdown of SLK by siRNA. MEF3T3 cells were transfected with control (50 nM) or SLK specific siRNA (25 and 50 nM) as described. Western blot analysis of treated lysates indicates that SLK siRNA at 50 nM resulted in a marked knockdown of SLK. Reprobing the membrane with an α-tubulin antibody was used as a control for loading (lower panel). (c) Effect of SLK knockdown on migration of MEF3T3 cells through a transwell chamber. Cells were treated with SLK-specific or control siRNA and assayed for migration through a chamber coated with bovine serum albumin (BSA) (10 mg/ml) (control) or fibronectin (FN) (10 μg/ml). Cells treated with SLK siRNA migrated 2-3 times less efficiently than control treated cells. (d) Expression of SLK kinase inactive delays microtubule-dependent adhesion turnover. Subconfluent MEF3T3 fibroblasts were infected with adenoviral constructs encoding kinase-defective SLK (AdHA-KΔC) or GFP control. Cultures were then treated with nocodazole (10 mM) for 4 h, washed and surveyed for FAK-pTyr397 levels over time. Expression of HA-tagged SLK was confirmed by Western blot analysis. Kinase-deficient SLK interferes with focal adhesion turnover as evidenced by the delayed disappearance of FAK-pTyr397.

FIG. 16 shows results suggesting that Ldb 1 and 2 associate with the ATH domain of SLK. (a) Ldb 1 and 2 bind the Gal4 DBD-SLK ATH fusion protein but not Gal4-DBD. Transformed yeast were plated on selective media (-Trp/-Leu/-His) and only cells expressing the Gal4-DBD-ATH fusion and Ldb1 or 2 Gal4-AD fusions grew on triple drop-out medium containing 20 mM 3-amino-triazole. Colonies were patched on selective media and assayed for β-galactosidase production. Only the Gal4DBD-ATH fusions showed β-galactosidase activity indicating that the ATH-Ldb association is direct. (b) Domain organization of the Ldb factors. Ldb proteins contain an amino-terminal dimerization domain (DD), a central domain containing a nuclear localization signal (NLS) and a carboxy-terminal LIM binding domain (LBD) (c) SLK deletion mutants of the ATH domain. Constructs consisted of SLK MNAP C-ter (aa 774-910), ATH-N (aa 881-994) and ATH-C (aa 981-1127). (d) SLK deletion mutants include the ATH domain (SLK 950-1202), a deletion lacking most of the ATH domain (SLK 1-950) or lacking half the MNAP and the entire ATH domain (SLK 1-551) and the kinase domain alone (SLK 1-373). (e) Binding of in vitro translated Ldb2 to GST-SLK deletions (see above). GST-SLK fusions were incubated with [35S]-labeled Ldb2, washed and bound Ldb2 was detected by SDS-PAGE and autoradiography. Ldb2 binds preferentially to the SLK ATH domain in vitro supporting a direct interaction between Ldb2 and SLK. (f) Mapping of the Ldb2 binding domain within the ATH domain of SLK. SLK deletion mutants encompassing the MNAP and ATH domains (see 16c) were assayed for binding to Ldb2. Ldb2 was in vitro translated ([35S]) and incubated with GST-MNAP C-terminus (MNAP-C), GST-ATH N-terminus (ATH-N) and GST-ATH C-terminus (ATH-C) or GST alone. Ldb2 bound almost exclusively to GST-ATH-N indicating that Ldb2 binds preferentially to this region of the SLK ATH domain. Identical results were obtained with Ldb1 (not shown).

FIG. 17 shows results suggesting that SLK and Ldb associate in vitro and in vivo. (a) Schematic representation of Ldb2 deletion constructs. Full length Ldb2 (Ldb2 FL; full length) and various deletion mutants are indicated including the Ldb2 LIM binding domain (LBD; aa 298-373), Ldb2 lacking the LIM binding domain (Ldb2 ΔLBD; aa 1-296), versions of the Ldb2 dimerization domain (Ldb2 DD; aa 1-186 and DDΔC; aa 1-24) and the Ldb2 nuclear localization signal domain (Ldb2 NLS; aa 188-288). (b) Association of Ldb2 deletion mutants with the SLK ATH domain. Ldb2 mutants were translated in the presence of [35S] methionine and subjected to a binding assay with GST-ATH as described. The Ldb2 dimerization domain and the NLS domain could independently bind the SLK ATH domain suggesting a strong interaction between the two factors. No binding was observed to the LIM-binding domain. (c) Schematics of Myc epitope-tagged SLK deletion mutants. Full length SLK (SLK), the kinase domain alone (SLK 1-373), SLK lacking the kinase domain (SLK 373-1202) and the SLK ATH domain (SLK 950-1202) are shown. (d) Co-transfection of HA-tagged Ldb2 and Myc-tagged SLK deletion constructs was performed in HEK 293 cells. Extracts were immunoprecipitated with anti-HA antibody (12CA5) and immunoblotted with anti-Myc antibody (9E10) (bottom panel). Expression of the transfected constructs was confirmed by western blotting (top and middle panels). Only Myc-SLK373-1202 and Myc-SLK950-1202 co-precipitated with HA-Ldb2 suggesting that the ATH domain is required and that the kinase domain interferes with anti-HA co-precipitation of Myc-SLK and HA-Ldb2. (e) Reciprocal co-IP showing that full length HA-SLK associates with Myc-Ldb1 and 2. (f) Association of endogenous SLK and Ldb. Ldb1 was immunoprecipitated from protein lysates obtained from MEF cells and primary neurons and subjected to western blot analysis with an anti-SLK antibody. A 220 KDa band corresponding to SLK co-precipitated with Ldb2. Reciprocal immunoprecipitations were also performed on N1E 115 neuroblastoma cells or primary cortical neurons. A 50 Kda band corresponding to Ldb1 co-precipitated with SLK. As a control for non-specific binding to the sepharose beads, protein-A sepharose beads alone were added to 400 μg of the same protein lysates. Similar results were obtained with Ldb2

FIG. 18 shows results confirming detection of SLK, Ldb2 and Ldb1 in leading edge ruffles and microtubules. Co-localization of Rac1 (d), Paxillin (e) and Ldb1 (f) with SLK (a-c) in membrane ruffles of migrating fibroblasts on fibronectin (arrowhead). Note the absence of SLK in mature adhesions identified by Paxillin staining (e; arrow). Double staining with SLK (g) or Ldb2 (j) with α-tubulin (h and k) shows that they can be co-localized to microtubules in leading edge ruffles. (m) Ldb2 binds α-tubulin. GST pulldown assays shows that Ldb2 but not Ldb1 can directly interact with α-tubulin.

FIG. 19 shows results indicating that deletion of Ldb1 results in complex disruption and enhanced migration. (a-left panel) MEFs obtained from floxed-Ldb1 mice were generated and infected with recombinant adenovirus harbouring a GFP or Cre recombinase cDNA. Expression of Cre recombinase resulted in a marked decrease in Ldb1 levels with little effect on Ldb2, SLK or PKR (control). (right panel) SLK was immunoprecipitated from lysates obtained from Ad-GFP or Ad-Cre infected cells. SLK precipitation efficiency and kinase activity was consistently greater in lysates from Cre-infected cells. In addition, Ldb2 association with the SLK complex was lost in cells lacking Ldb1 (bottom panel). (b) Ldb2 interacts with SLK kinase domain and ATH. GST-SLK1-373 pull downs show that Ldb1 can weakly interact with the SLK kinase domain. When co-expressed in 293, His-Ldb1 increases the efficiency of co-precipitation of the SLK kinase domain with the ATH region, suggesting that Ldb1 can facilitate “bridging” of those domains. (c) Ldb1/2 inhibit SLK kinase activity in vitro. Recombinant GST-tagged SLK was prepared and mixed with recombinant His-tagged Ldb2 (lane 2), His-tagged Ldb1 (lane 3) or both (lane 4) and assayed for kinase activity on histone H1. Addition of recombinant Ldb proteins inhibited SLK activity (d) Activation of SLK by scratch wounding is accompanied by a conformational change. Endogenous SLK complexes were immunoprecipitated from unscratched MEFs or 60 min post-wounding and subjected to trypsin digestion. Differential trypsin sensitivity was observed indicative of altered conformation.

FIG. 20 shows results that Ldb2 or Ldb1 knock down increases cell motility. (a) NIH3T3 cells were treated with control siRNA or siRNAs to Ldb1, 2, or both and assayed for protein levels by western blot analysis. Efficient knock down of Ldb1 was achieved. (b) The same cultures were analyzed for migration potential in a transwell migration assay. Reduction of either Ldb1 or Ldb2 levels enhanced cell motility. Similarly, deletion of Ldb1 in “floxed”-Ldb1 MEF increased cell motility. (c) Ldb1 knock down increase microtubule-dependent adhesion turnover. NIH 3T3 cells transfected with Ldb1 siRNAs were subjected to nocodazole washouts and analysed for FAK-pTyr397 levels. The kinetics of turnover in Ldb1 knock downs were faster than that observed in control cultures as evidenced by focal adhesion re-assembly at 45 minutes.

FIG. 21 shows results of upregulation of SLK activity by Neu-NT and LMO4. (A) Co-transfection of HA-SLK and wildtype (WT) or activated Neu (NeuNT) in HeLa cells results in SLK kinase activation as assessed by in vitro kinase assay. HA-SLK is the wildtype kinase and K63R is the kinase inactive mutant. Probing of whole cell lysates (WCL) shows overexpression of NeuNT. Reprobing of the IP for SLK shows equal efficiencies. A survey of the IP for ErbB2 revealed that it did not co-precipitate with SLK. (B) Stimulation of Sk-Br-3 cells with 1 nM Heregulin for 30 min results in SLK activation as assayed by IP kinase assays, suggesting that human SLK is also activated in breast carcinoma by HRG stimulation. (C) Western blot analysis showing expression of ErbB2 in T47D and the 5R clone. The ErbB2 receptor in the 5R clone remains in the ER and cannot be activated at the membrane as shown by the more rapidly migrating unphosphorylated species. (D) Transfection of SLK siRNA in a murine breast carcinoma line (NMuMG) expressing activated ErbB2 (NT) or a signaling deficient receptor (PD) inhibits the chemotactic response to heregulin beta in vitro.

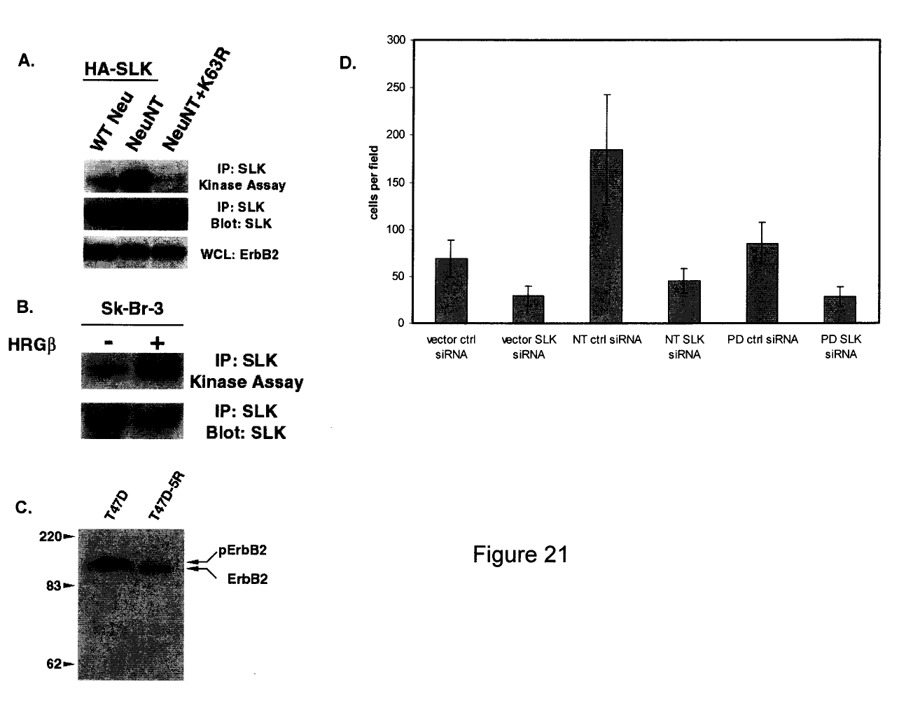

FIG. 22 shows results that DN-SLK interferes with HRG-induced motility and induces SLK recruitment into lamellipodia. (A) Expression of SLK in human breast carcinoma lines. All lines tested expressed similar levels of endogenous SLK. (B) MCF-7 cells expressing retrovirally transduced (HA-KΔC) DN-SLK prior to migration assays. (C) and (D) HRG-stimulated motility is impaired in Sk-Br-3 cells (C) or MCF-7 (D) expressing DN-SLK. Cells were plated in the top chamber in serum free medium and stimulated overnight with 1 nM HRG in the bottom chamber. Cells that migrated on the underside were visualized by DAPI staining and enumerated. (E) Similarly, SLK inhibits HRG-induced chemotaxis in T47D breast carcinomas. The 5R clone which does not express surface ErbB2 did not exhibit a significant response.

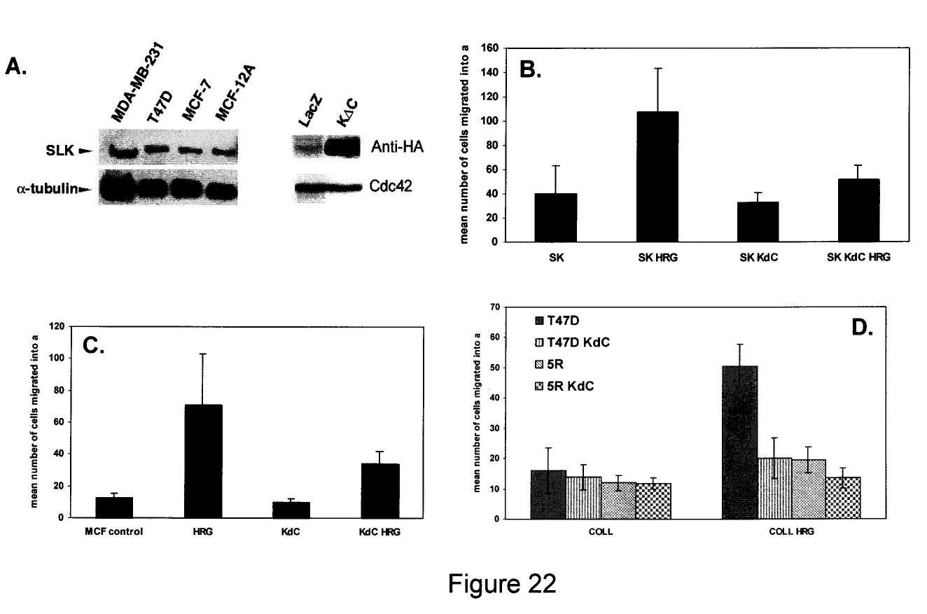

FIG. 23 shows results suggesting the association and inhibition of SLK by nm23 in vivo. (A) NMuMg breast carcinoma were scratch wounded and assayed for SLK kinase activity and nm23 association. SLK activation results in loss of nm23 binding. (B) SLK kinase activity is downregulated by overexpression of nm23. (C) Activation of SLK by ErbB2 receptor requires tyrosine 1201 (YC), 1227 (YD) and 1253 (YE). These are Crk/Shc/PLCg, Shc and DOK binding sites, respectively.

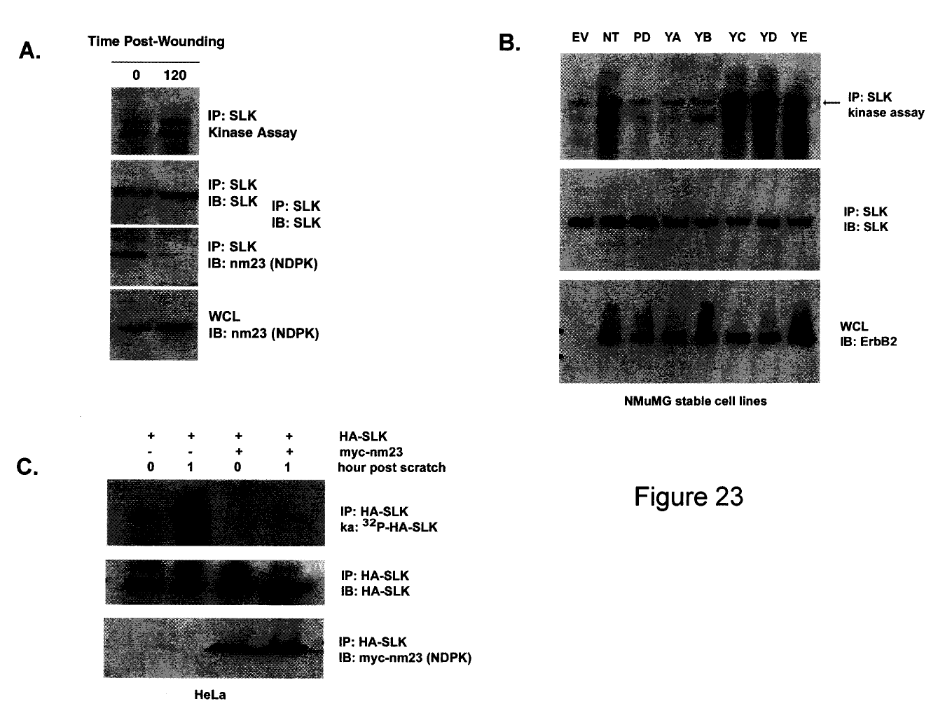

### DESCRIPTION OF PREFERRED EMBODIMENT

The present invention relates to kinase inhibitors. More specifically, the present invention relates to inhibitors of Step 20-like kinase (SLK) and methods of modulating cell cycle progression or cell motility using such inhibitors.

The following description is of a preferred embodiment by way of example only.

According to an embodiment of the present invention, there is provided an inhibitor of Ste20-like kinase (SLK).

By “inhibitor of Ste20-like kinase (SLK)” or “SLK inhibitor” it is meant a product, compound or composition that is capable of interfering with the normal kinase activity of SLK. Various inhibitors of SLK are contemplated by the present invention, for example, but not limited to antibodies or fragments thereof that bind to and inhibit the activity of SLK, antisense or siRNA nucleic acids that for example, but not limited to downregulate SLK production thereby inhibiting SLK activity, catalytically inactive SLK proteins, variants or fragments of SLK that act for example, but not limited to, act as dominant negatives or molecular decoys for protein binding partners of SLK, or small molecules that interfere with the biological activity of SLK.

Without wishing to be considered limiting in any manner, and as will be appreciated by a person of skill in the art, an SLK inhibitor that reduces the production of SLK protein, sequesters, binds or stabilizes SLK in an in-active conformation, promotes increased degradation of SLK protein or that inhibits interaction of SLK with normal binding partners necessary for signal transduction may be considered an inhibitor of Ste20-like kinase (SLK) provided it interferes with the normal kinase activity of SLK. It is to be understood that the SLK inhibitors as described herein may or may not affect the specific activity of an endogenous SLK enzyme present in a cell. The present invention is meant to include all such inhibitors.

Preferably, the SLK inhibitor reduces endogenous SLK kinase activity by at least about 20%, more preferably at least about 30%, still more preferably at least about 40%, 50%, 60%, 70%, 80%, 90% or 100% compared to wild-type SLK. Further, the SLK inhibitors of the present invention may exhibit a range of activity inhibition as defined by any two of the values listed above. In an alternate embodiment, the SLK inhibitor reduces the specific activity of SLK by about 20%, more preferably about 30%, still more preferably about 40%, 50%, 60%, 70%, 80%, 90%, or about 100% compared to wild-type SLK. Further, the SLK inhibitors of the present invention may exhibit a range of specific activity inhibition defined by any two of the values listed above.

SLK kinase activity may be determined using any suitable assay known in the art, for example, but not limited to, as described in Example 1 under the section “Western Blotting, Immunoprecipitations and in vitro Kinase Assays” or the kinase assay as provided herein (30; which is incorporated by reference). Alternatively, SLK kinase activity may be assayed by flow cytometric analysis as described herein, wherein an inhibitor of SLK reduces or inhibits cell cycle progression as compared to a control. For example, but not wishing to be limiting, expression of a kinase inactive SLK results in a G₂/M block in cells, whereas control cells continue to cycle. For comparative purposes, a wild-type SLK comprises the SLK protein sequence defined by SEQ ID NO:1.

The SLK inhibitor may comprise a catalytically inactive SLK protein, fragment or variant of a wild-type protein. For example, but not to be considered limiting in any manner, the catalytically inactive SLK may be prepared by mutating one or more amino acids required for kinase activity. Also, catalytically inactive variants of SLK may be prepared by deleting one or more polypeptide segments required for kinase activity, for example, entire protein regions or domains responsible for kinase activity may be deleted or mutated. Examples of catalytically inactive SLK proteins are known in the art and are described herein. It is preferred that SLK inhibitors exhibit no kinase activity. However, it is contemplated that the inhibitor may exhibit reduced kinase activity, that is, less than that of wild-type SLK. In such embodiments, preferably the SLK kinase exhibits less than about 50% of wild-type SLK activity, more preferably less than about 40%, 30%, 20% and still more preferably less than about 10% of wild-type SLK kinase activity.

The SLK inhibitor also may comprise one or more fragments of a wild-type SLK that interfere with the normal kinase activity of the protein. For example, but not to be considered limiting, or bound by theory, a fragment of SLK may bind to one or more protein binding partners of SLK required for normal signal transduction activity. Accordingly, an SLK inhibitor may comprise a fragment of SLK, for example, a polypeptide comprising 5, 7, 9, 10, 12, 15, 17, 20, 21, 25, 30, 35, 50, 60, 70, 80, 90, 100, 110, 120, 130, 140, 150, 160, 170, 180, 190, 200, 300, 400, 500, 600, 700, 800, 900, 1000, 1100, 1200 or more amino acids of SLK. Further, SLK inhibitors that are fragments of SLK may be characterized as comprising a length defined by a range of any two of the values listed.

An SLK inhibitor may also comprise one or more compounds that interact with a wild-type SLK and inhibit kinase activity. In an embodiment, which is not meant to be limiting, the SLK inhibitor binds to wild-type SLK. For example, but not to be considered limiting in any manner, the SLK inhibitor may comprise myosin binding protein (mybp-c), CLIM1/lbd2, CLIM2/Ldb1, nm23 or a fragment thereof. In an alternate embodiment, the inhibitor of SLK may comprise an antibody, or a fragment thereof that is capable of binding to SLK and inhibiting cell cycle proliferation.

Without wishing to be limiting in any manner, a representative wild-type SLK kinase is described under genbank accession number AAD28717 (SEQ ID NO:1). Other variants of wild-type SLK kinase sequences from different species are also known in the art and/or can be readily identified, for example, by performing a blast search using SEQ ID NO:1 or a fragment thereof. In a preferred embodiment, the SLK inhibitor is a human SLK protein that is kinase dead. Thus, the present invention contemplates mutants of the above known SLK kinases which are substantially identical to SEQ ID NO:1 but that are kinase deficient, more preferably kinase dead. For example, but not wishing to be considered limiting, the present invention contemplates inhibitors of SLK kinase that comprise one or more mutations that abolish ATP binding, for example, but not to be considered limiting, the K63R mutation (relative to the amino acid sequence defined by SEQ ID NO:1). Such a sequence is shown in SEQ ID NO:2. Other SLK inhibitors having the corresponding lysine (K) mutated to any other amino acid are also contemplated, as are fragments and variants thereof which inhibit endogenous SLK activity in a cell. By way of example, the present invention contemplates SLK inhibitors that are kinase dead and that exhibit between about 70% and 100% identity with SEQ ID NO:1, for example, but not limited to 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% sequence identity with SEQ ID NO:1.

An SLK inhibitor also may comprise a fusion polypeptide comprising an SLK inhibitor and a heterologous polypeptide sequence. In a preferred embodiment, which is not meant to be limiting, there is provided a fusion protein or fusion polypeptide comprising an SLK inhibitor and a protein transduction domain. By “protein transduction domain” it is meant a sequence of nucleic acids that encode a polypeptide, or a sequence of amino acids comprising the polypeptide, wherein the polypeptide facilitates localization to a particular site, for example a cell or the like, or it may facilitate transport across a membrane or lipid bilayer. The polypeptides and nucleic acids of the present invention may be fused to a protein transduction domain to facilitate transit across lipid bilayers or membranes.

Many polypeptides and nucleic acids do not efficiently cross the lipid bilayer of the plasma membrane, and therefore enter into cells at a low rate. However, there are certain naturally occurring polypeptides that can transit across membranes independent of any specific transporter. Antennapedia (Drosophila), TAT (HIV) and VP22 (Herpes) are examples of such polypeptides. Fragments of these and other polypeptides have been shown to retain the capacity to transit across lipid membranes in a receptor-independent fashion. These fragments, termed protein transduction domains, are generally 10 to 27 amino acids in length, possess multiple positive charges, and in several cases have been predicted to be ampipathic. Polypeptides and nucleic acids that are normally inefficient or incapable of crossing a lipid bilayer, can be made to transit the bilayer by being fused to a protein transduction domain.

U.S. Publication 2002/0142299 (which is incorporated herein by reference) describes a fusion of TAT with human beta-glucuronidase. This fusion protein readily transits into various cell types both in vitro and in vivo. Furthermore, TAT fusion proteins have been observed to cross the blood-brain-barrier. Frankel et al. (U.S. Pat. No. 5,804,604, U.S. Pat. No. 5,747,641, U.S. Pat. No. 5,674,980, U.S. Pat. No. 5,670,617, and U.S. Pat. No. 5,652,122; which are incorporated herein by reference) have also demonstrated transport of a protein (beta-galactosidase or horseradish peroxidase) into a cell by fusing the protein with amino acids 49-57 of TAT.

PCT publication WO01/15511 (which is incorporated herein by reference) discloses a method for developing protein transduction domains using a phage display library. The method comprises incubating a target cell with a peptide display library and isolating internalized peptides from the cytoplasm and nuclei of the cells and identifying the peptides. The method further comprised linking the identified peptides to a protein and incubating the peptide-protein complex with a target cell to determine whether uptake is facilitated. Using this method a protein transduction domain for any cell or tissue type may be developed. US Publication 2004/0209797 (which is incorporated herein by reference) shows that reverse isomers of several of the peptides identified by the above can also function as protein transduction domains.

PCT Publication W099/07728 (which is incorporated herein by reference) describes linearization of protegrin and tachyplesin, naturally occurring as a hairpin type structure held by disulphide bridges. Irreversible reduction of disulphide bridges generated peptides that could readily transit cell membranes, alone or fused to other biological molecules. US Publication 2003/0186890 (which is incorporated herein by reference) describes derivatives of protegrin and tachyplesin that were termed SynB1, SynB2, SynB3, etc. These SynB peptides were further optimized for mean hydrophobicity per residue, helical hydrophobic moment (amphipathicity), or beta hydrophobic moment. Various optimized amphipathic SynB analog peptides were shown to facilitate transfer of doxorubicin across cell membranes. Further, doxorubicin linked to a SynB analog was observed to penetrate the blood-brain-barrier at 20 times the rate of doxorubicin alone.

The protein transduction domains described in the preceeding paragraphs are only a few examples of the protein transduction domains available for facilitating membrane transit of small molecules, polypeptides or nucleic acids. Other examples are transportan, W/R, AlkCWK18, DipaLytic, MGP, or RWR. Still many other examples will be recognized by persons skilled in the art. The SLK inhibitors of the present invention may employ any of such sequences.

An SLK inhibitor may comprise a nucleic acid encoding one or more of the SLK inhibitors described above. In an embodiment the SLK inhibitor is a nucleic acid encoding a catalytically inactive SLK, a variant or fragment thereof that interferes with SLK kinase activity. Also contemplated, the SLK inhibitor may comprise an SLK antisense nucleic acid or fragment thereof, for example, but not limited to antisense DNA, antisense RNA, a short interfering nucleic acid, for example, but not limited to siRNA. In a preferred embodiment, the SLK antisense nucleic acid comprises a short interfering RNA (siRNA), RNAi or duplex thereof.

In an embodiment that is not meant to be limiting in any manner, the nucleic acid encodes the SLK inhibitor defined by SEQ ID NO:2.

To determine whether a protein or nucleic acid exhibits identity with the sequences presented herein, oligonucleotide or protein alignment algorithms may be used, for example, but not limited to a BLAST (GenBank URL: www.ncbi.nlm.nih.gov/cgi-bin/BLAST/, using default parameters: Program: blastn; Database: nr; Expect 10; filter: default; Alignment: pairwise; Query genetic Codes: Standard(1)), BLAST2 (EMBL URL: http://www.embl-heidelberg.de/Services/index.html using default parameters: Matrix BLOSUM62; Filter: default, echofilter: on, Expect: 10, cutoff: default; Strand: both; Descriptions: 50, Alignments: 50), or FASTA, search, using default parameters. Polypeptide alignment algorithms are also available, for example, without limitation, BLAST 2 Sequences (www.ncbi.nlm.nih.gov/blast/bl2seq/bl2.html, using default parameters Program: blastp; Matrix: BLOSUM62; Open gap (11) and extension gap (1) penalties; gap x_dropoff: 50; Expect 10; Word size: 3; filter: default).

An alternative indication that two nucleic acid sequences are substantially identical is that the two sequences hybridize to each other under moderately stringent, or preferably stringent, conditions. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. for at least 1 hour (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. for at least 1 hour (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, but not wishing to be limiting, stringent conditions are selected to be about 5° C. lower than the thermal melting point for the specific sequence at a defined ionic strength and pH.

The present invention also contemplates nucleic acids which hybridize under high stringency conditions to a nucleic acid molecule which encodes an SLK kinase, preferably the human SLK. Appropriate stringency conditions which promote DNA hybridization are known to those skilled in the art, or can be found in several reference documents, for example, but not limited to Current Protocols in Molecular Biology, John Wiley & Sons, N.Y. (1989), which is herein incorporated by reference.

SLK inhibitors that are nucleic acids may comprise part of a larger nucleic acid or genetic construct, for example, but not limited to a vector or the like. In an embodiment, which is not meant to be limiting, an SLK inhibitor is produced in a cell by infecting the cell with a virus genetically engineered to produce the SLK inhibitor. In a preferred embodiment, but without wishing to be limiting, the SLK inhibitor may be encoded by a virus, for example, but not limited to an adenovirus, lentivirus, retrovirus or the like. In a preferred embodiment, the virus is an adenovirus or retrovirus.

The nucleotide sequence may be operably linked to regulatory elements in order to achieve preferential expression at desired times or in desired cell or tissue types. Furthermore, as will be known to one of skill in the art, other nucleotide sequences including, without limitation, 5′ untranslated region, 3′ untranslated regions, cap structure, poly A tail, translational initiators, sequences encoding signalling or targeting peptides, translational enhancers, transcriptional enhancers, translational terminators, transcriptional terminators, transcriptional promoters, may be operably linked with the nucleotide sequence encoding a polypeptide (see as a representative examples “Genes VII”, Lewin, B. Oxford University Press (2000) or “Molecular Cloning: A Laboratory Manual”, Sambrook et al., Cold Spring Harbor Laboratory, 3rd edition (2001)). A nucleotide sequence encoding a SLK inhibitor or a fusion polypeptide comprising a SLK inhibitor and a protein transduction domain may be incorporated into a suitable vector. Vectors may be commercially obtained from companies such as Stratagene or InVitrogen. Vectors can also be individually constructed or modified using standard molecular biology techniques, as outlined, for example, in Sambrook et al. (Cold Spring Harbor Laboratory, 3rd edition (2001)). A vector may contain any number of nucleotide sequences encoding desired elements that may be operably linked to a nucleotide sequence encoding a polypeptide or fusion polypeptide comprising a protein transduction domain. Such nucleotide sequences encoding desired elements, include, but are not limited to, transcriptional promoters, transcriptional enhancers, transcriptional terminators, translational initiators, translational, terminators, ribosome binding sites, 5′ untranslated region, 3′ untranslated regions, cap structure, poly A tail, origin of replication, detectable markers, affinity tags, signal or target peptides and the like. Persons skilled in the art will recognize that the selection and/or construction of a suitable vector may depend upon several factors, including, without limitation, the size of the nucleic acid to be incorporated into the vector, the type of transcriptional and translational control elements desired, the level of expression desired, copy number desired, whether chromosomal integration is desired, the type of selection process that is desired, or the host cell or the host range that is intended to be transformed.

The SLK inhibitors described herein, as well as SLK inhibitors known in the art may be employed in a method to reduce or inhibit cell proliferation. For example, the present invention provides a method for inhibiting the proliferation of a cell by inhibiting endogenous SLK activity in the cell. In an alternate embodiment, there is provided a method of inhibiting the proliferation of a cancer or tumor cell by inhibiting endogenous SLK activity in the cell. Methods to reduce or inhibit cell proliferation may be practised in vitro or in vivo. In a preferred embodiment, the method is practised in a subject, preferably a human subject.

The present invention also contemplates a method of inhibiting the movement or migration of a cell by inhibiting endogenous SLK activity in the cell. In a further embodiment, there is provided a method of preventing a cancer cell or tumor cell from metastasizing by inhibiting endogenous SLK activity in the cancer or tumor cell. In a preferred embodiment, the method is practised in a subject, preferably a human subject.

The present invention also contemplates a method of inhibiting the proliferation of a cancer or tumor cells in a subject by administering an SLK inhibitor. The subject may be an animal subject, preferably a mammalian subject. In a preferred embodiment, the subject is a human subject.

Various types of cancers and tumors may be treated using the SLK inhibitors and methods as described herein, including those types of cancers with a high propensity to metastasize. Without wishing to be limiting, the present invention contemplates treating cancers including, but not limited to breast, bone, brain, blood including any blood cell type cancer, prostate, liver, kidney, skin, stomach, spleen, colon, rectal, testicular, ovarian, uteral, thyroid, and the like. Accordingly, the present invention contemplates a method for treating a cancer or tumour in a subject comprising the step of administering an SLK inhibitor to the subject in need thereof. Further, the present invention contemplates a method of preventing metastasis of a cancer comprising the step of administering an SLK inhibitor to subject in need thereof. The invention also contemplates a method of preventing proliferation of a cancer or tumour in a subject, comprising the step of administering an SLK inhibitor to a subject in need thereof.

The present invention also contemplates a cell, group of cells, tissue or the like that comprises the SLK inhibitors as described herein. In an embodiment, which is not meant to be limiting, the cell is a non-cultured cell, preferably an in-vivo cell. In a further embodiment, the cell is a cancer cell or a tumour cell, more preferably an in vivo cancer cell or tumour cell. The present invention also contemplates cancers that are carcinomas and sarcomas. In still a further embodiment, the cell is a HER2+ carcinoma. In all of such cases, it is preferred the SLK inhibitor is a heterologous SLK inhibitor that does not exist naturally in the cell. However, the present invention also contemplates artificially increasing the presence of a naturally occurring SLK inhibitor in a cell, for example, but not limited to by overexpressing the a naturally occurring SLK inhibitor in the cell.

All citations are hereby incorporated by reference.

### EXAMPLES

### Example 1

### Ste20-like Kinase SLK is Required for Normal Cell Cycle Progression

### Experimental Procedures

Cell Lines, Culture, and Adenovirus Infection—The mouse fibroblast lines MEF-3T3 (MEF Tet-Off, C3018, Clontech) and C3H10T½ (ATCC number CCL-226) were employed in experiments. Similar results were obtained for both cell lines. The GFP-tubulin expressing cells (LLCKP-1) were a kind gift from Patricia Wadsworth (34). Cell lines were maintained at 37° C. with 5% CO₂ in Dulbecco's modified Eagle's medium (DMEM, Bio-Whitaker) supplemented with 10% fetal bovine serum (FBS), 2 mM L-glutamine, 50 μg/mL penicillin, and 50 μg/mL streptomycin. For cell cycle experiments, fibroblasts were arrested by 48 hour incubations in 0.25% FBS-DMEM and released from quiescence by the addition of 20% FBS-DMEM. The epitope-tagged kinase dead or activated versions of SLK used in these studies (HA-KΔC or HA-YΔC) have been previously described (1) and consist of a C-terminal truncation (aa 1-373) with or without an ATP-binding site (Lys 63->Arg) mutation. To monitor the effect of kinase deficient SLK on cell cycle kinetics, adenoviral vectors expressing HA-KΔC or a β-galactosidase (LacZ) control were used to infect quiescent cultures. Cells were infected at a MOI of 100 by the addition of the adenovirus directly to cells in 0.25% FBS-DMEM 16 hours before the addition of 20% FBS-DMEM and analysis. To isolate fibroblast populations synchronized at M phase, the cultures were treated overnight with nocodazole (40 ng/mL, Sigma-Aldrich) and shaken off into PBS by aggressive tapping against a solid surface. Detached cells floating in PBS were then collected by centrifugation.

For mitotic spindle induction experiments, LLCKP-1 cells expressing GFP-tubulin were microinjected with HA-YΔC expression plasmid as described previously (1). Xenopus oocytes were prepared and injected with cRNA as described (35). Germinal vesicle breakdown (GVBD) was determined from at least 20 oocytes pooled from two animals 15 hours following SLK injection or progesterone treatment. Western blot analysis for Myc-SLK was performed 15 hours post-injection.

Western Blotting, Immunoprecipitations, and in vitro Kinase Assays—For protein analysis, the cells were lysed in modified RIPA buffer as previously described (1). Equal amounts of extracts were resolved by SDS-PAGE and transferred to polyvinylidene difluoride (PVDF) membranes (Perkin Elmer Life Sciences Inc.) which were then probed with the following antibodies. Anti-Cdc2, cyclins D, E, A, and B (Upstate Biotechnology). The SLK antibody was as previously described (30). Primary antibodies were detected using either a goat anti-rabbit IgG or goat anti-mouse IgG horseradish peroxidase-labeled secondary antibody (BioRad) and visualized using Western Lightning chemiluminescence reagent (Perkin Elmer Life Sciences Inc.) and exposure to X-ray film. SLK immunoprecipitations and kinase assays were carried out essentially as previously described (31). Briefly, equal amounts of lysate were immunoprecipitated for 2 hours at 4° C. using 1 μg of SLK antibody and 20 μL of protein A sepharose (4 fast flow, Amersham Biosciences). Immunoprecipitates were then washed three times with NETN (50 mM Tris-HCl [pH 7.5], 150 mM NaCl, 1 mM EDTA, 0.1% Nonidet P-40) and once with kinase buffer (20 mM Tris-HCl [pH 7.5], 15 mM MgCl₂, 10 mM NaF, 10 mM β-glycerophosphate, 1 mM orthovanadate). Kinase reactions (20 μL) were then initiated by the addition of 5 μCi of [32P]ATP and incubated at 30° C. for 30 minutes. These reactions were terminated by the addition of 4×SDS loading buffer and then resolved by SDS-PAGE, transferred to PVDF membranes, and exposed to X-ray film. Membranes were then probed for SLK to assess immunoprecipitation efficiency. SLK autophosphorylation was used as an indicator of kinase activity.

Immunofluorescence and Flow Cytometry—Immunofluorescence studies were carried out by fixation of cells growing on glass cover slips in 4% paraformaldehyde for 10 minutes. The cells were then washed twice with PBS and incubated with primary antibodies for 1 hour. The primary antibodies used in immunofluorescence studies were as follows: anti-α-tubulin(clone DM1, Sigma), anti-phospho-H3 (Ser10) (Cell Signaling Technology), anti-beta-galactosidase (Promega), anti-HA (12CA5 or sc-805, Santa Cruz Biotechnology Inc.) and anti-SLK (30). Antibodies were detected with either anti-mouse or anti-rabbit antibodies conjugated to either fluorescein isothiocyanate (FITC) or tetramethyl rhodamine isothiocyanate (TRITC) (Sigma). DAPI (4′, 6-diamidino-2-phenylindole, 0.25 μg/mL) was used to stain DNA and the samples were visualized with a Zeiss Axioscope100 epifluorescence microscope equipped with the appropriate filters and photographed with a digital camera (Sony Corporation HB050) using the Northern Eclipse software package. Quantitative analysis for phospho-H3 (pH3) immunostaining was performed by visually scoring stained cells for both HA (or LacZ) and pH3. The data were graphed as double positive (HA or LacZ+pH3) cells for each time point analysed. At least 200 HA or LacZ-positive cells were scored for each time point. Cells analysed by flow cytometry were trypsinized and washed once in 10% FBS-DMEM. Cells were then washed twice in PBS supplemented with 1 mM EDTA (PBSE) and then fixed in 1 mL of PBSE by the drop wise addition of 2 mL of 80% ethanol pre-chilled to −20° C. The samples were then stored at −20° C. for a minimum of 2 hours, washed once in PBSE, resuspended in DNA content staining buffer (1.1% citrate buffer, 10 μg/mL propidium iodide, 1 mg/ml RNase) and incubated for 30 minutes at 37° C. before being analyzed on a Beckman-Coulter flow cytometer using the Expo 32 software package. BrdU pulse labeling was performed using a BrdU Flow Kit (BD Biosciences) according to the manufacturers instructions. Briefly, 16 hours before labeling, cultures were infected with either HA-KΔC or LacZ adenovirus at a MOI of 100 for 90 minutes in unsupplemented DMEM and then grown in 10% FBS-DMEM. The cultures were then labeled with BrdU for 1 hour and collected at various times. The DNA content of BrdU positive cells was then analyzed flow cytometrically.

Cell Counts, Cloning, and Transfections—To monitor cell proliferation, the cells were counted after infection on day 0 at a MOI of 100 as described above. Cell populations were trypsinized and scored by trypan blue exclusion over time. Cell counts were performed in triplicates in three independent experiments. For anti-sense SLK expression, a 5-prime 300 base pair SLK fragment was cloned into the pEMSV-puro expression vector in the reverse orientation. The antisense SLK plasmid and the puromycin control were then transfected into MEF-3T3 cells using LipofectAMINETM 2000 (Invitrogen) according to the manufacturers protocol and selected in puromycin (100 μg/mL) over a two-week period. Following selection, stable transfectants were visualized using CYTO-QUIK staining (Fisher Health Care) followed by several PBS washes. SLK Smart Pools siRNAs were obtained from Dharmacon against the following murine SLK target sequences: 5′-GGTTGAGATTGACATATTA-3′(SEQ ID NO:3). In addition to a scrambled siRNA (5′-GATAATTTATGGATGTGAC-3′) (SEQ ID NO:4), control siRNA comprised in the siCONTROL (Dharmacon; 5′-UAGCGACUAAACACAUCAAUU-3′) (SEQ ID NO:5), having no perfect match to known human or mouse sequences. All siRNAs were transfected using the Transit-TKO reagent (Mirrus Corp.) according to the manufacturer's instructions.

### Results

SLK Colocalizes with the Mitotic Spindle and is Regulated During the Cell Cycle

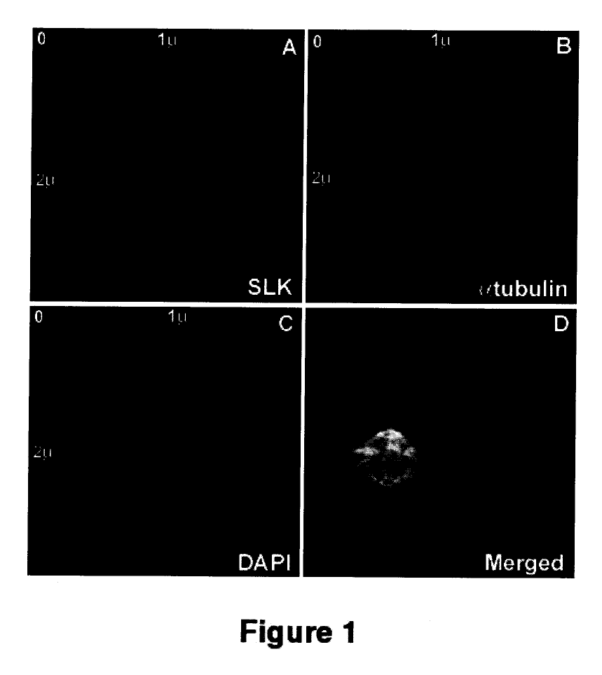

We have previously shown that a proportion of SLK is associated with the microtubule network of exponentially growing fibroblasts (1). During immunofluorescence studies involving the colocalization of SLK to the microtubule network in asynchronous cultures, we observed rare patterns of SLK and α-tubulin colocalization that resembled the mitotic spindle. To further investigate the possibility that SLK might co-localize with the mitotic spindle, we performed confocal microscopy. Although some SLK was found outside of the mitotic spindle, confocal analysis of DAPIstained cells, in combination with anti-SLK and anti-α-tubulin, shows that most of the SLK staining co-localized with tubulin during metaphase (FIGS. 1A and B). The observation that SLK is associated with α-tubulin, a central component of the mitotic machinery, suggests a role for SLK during mitosis. Alternatively, SLK may be required for spindle assembly.

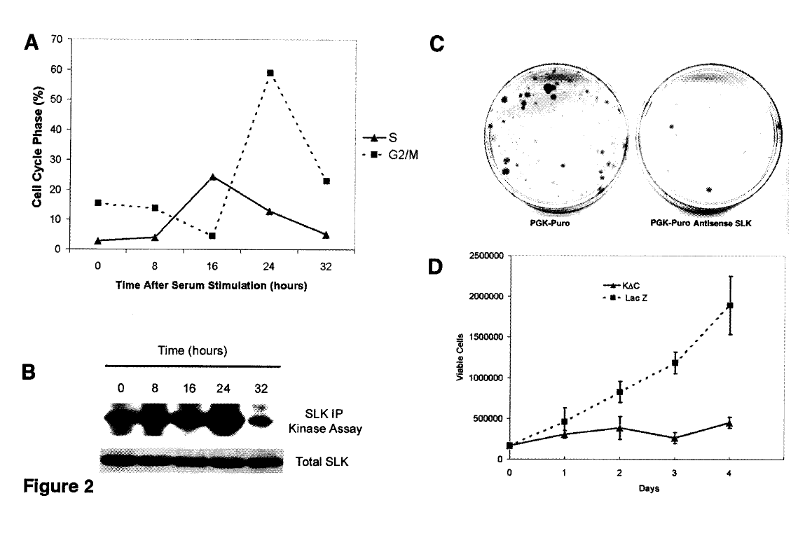

To further investigate the potential role of SLK in cell cycle progression, its activity was monitored throughout the different phases of the cell cycle using synchronized cell populations. Serum-starved cultures were released from G₀ by the addition of serum, collected at various times, and monitored for SLK activity and DNA content by kinase assays and FACS analysis, respectively. FIG. 2A displays the cell cycle phase as determined by flow cytometric measurements of DNA content in these synchronized populations following serum stimulation. After 24 hours of serum stimulation a marked and consistent 3-4-fold increase in SLK kinase activity (FIG. 2B) was observed when approximately 60-70% of the cells entered the G2/M compartment, as determined by FACS analysis. Similarly, an increase in SLK activity during G2 has previously been reported, albeit to a much lesser extent (˜1.3 fold) (33). Interestingly, as the cells exited M phase and re-entered G1, a marked reduction in kinase activity was observed. The total levels of SLK protein were found to be unaffected throughout the time course. These results suggest that SLK kinase activity is upregulated at a time point where the vast majority of the cells display a 4N DNA content.

### Kinase-Deficient SLK Inhibits Proliferation

To investigate the potential role of SLK in proliferation, an expression vector bearing an antisense SLK fragment was transfected into MEF-3T3 fibroblasts and subjected to puromycin selection. Stable clones were visualized after 14 days using Cyto-Quick stain (Fisher). As shown in FIG. 2C, antisense SLK-transfected cultures reproducibly displayed a marked reduction in colony numbers when compared to the pEMSV-puro control vector.

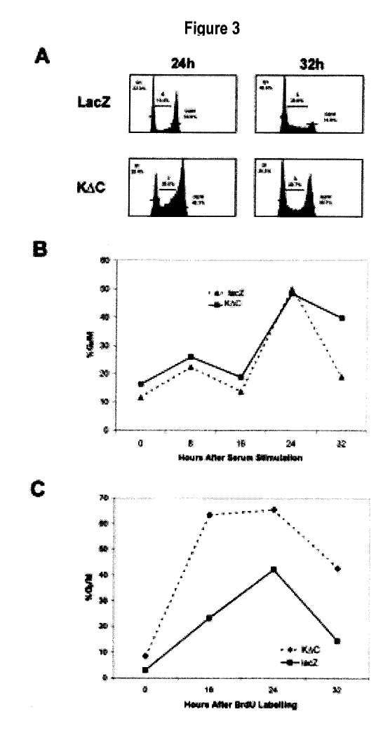

As for antisense SLK expression, infection of MEF-3T3 cells with an adenoviral vector carrying a kinase dead truncation of SLK (SLK1-373K63R, termed HA-KΔC) suppressed cell proliferation as determined by dye exclusion cell counts (FIG. 3). Taken together, these results indicate that SLK activity is required for cell proliferation and that our truncated kinase dead version, HA-KΔC, is able to interfere with SLKdependent pathways.

### SLK is Required for Progression Through G2

To further investigate the mechanism by which KΔC inhibits proliferation, quiescent MEF-3T3 cultures were infected with a KΔC-expressing adenovirus, or control LacZ virus, serum-stimulated and subjected to DNA content analysis over time. As shown in FIGS. 3A and B, cultures expressing LacZ proceeded through the cell cycle and exited the G2/M compartment by 32 h following serum stimulation. In contrast, KΔCexpressing cultures displayed a marked increase in the proportion of cells in the G2/M compartment, suggesting that KΔC expression delays progression through the G2 or M phase by inducing a specific block, or, alternatively, a failure to exit M phase.

Supporting these data, flow tracking of BrdU-pulsed exponentially growing KΔC-infected cultures shows that they proceed through the G2/M compartment with delayed kinetics when compared to LacZ-infected cells. Taken together, these results suggest that SLK is required for normal progression through the G2 or M phase.

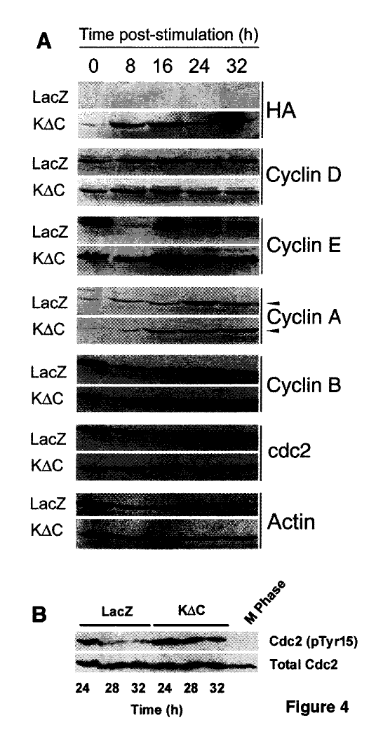

The levels of the various cyclins have been demonstrated to fluctuate during the cell cycle (reviewed in (5)). Cyclin D has been observed to be induced prior to S phase and to remain elevated in proliferating cells. Cyclin E is transiently upregulated at the G1/S boundary whereas cyclins A and B are induced at the S/G2 boundary and downregulated at the onset and the end of M phase, respectively. Therefore, to better define the cell cycle block induced by kinase deficient SLK, control and KΔC-infected cultures were surveyed for cyclin expression over time following release from Go. The infection efficiency was found to be typically between 70-80% for both viruses and the HA-KΔC protein was observed to be expressed at levels that were similar to endogenous SLK (data not shown). Our results show that, relative to actin, cyclin D was slightly upregulated following serum stimulation in both the control and KΔC-infected cultures, suggesting that they re-entered the cell cycle (FIG. 4A). Similarly, cyclin E levels were upregulated 16 hours following stimulation, when a significant proportion of the cells entered S phase (see FIG. 2A), and downregulated thereafter, suggesting that both cultures entered and exited S phase with similar kinetics. In addition, both cultures induced cyclin B expression at around 8 to 16 hours. Similarly, both cultures induced cyclin A at the G1/S transition. However, only control-infected cultures showed a marked and reproducible downregulation at 32 hours, suggesting that KΔCexpressing cells fail to downregulate cyclin A. Cyclin A degradation has been shown to occur during prometaphase, at the onset of mitosis (36,37). Therefore, our observations suggest that the KΔC-induced cell cycle block occurs in G2, prior to M phase. Without wishing to be bound by theory, one possibility is that the cell cycle block induced by KΔC fails to activate specific signals required for cyclin A degradation (36,37). Alternatively, SLK may be required for the direct activation of these checkpoints or proteolysis of cyclin A.

During interphase, cytosolic MPF is kept inactive by inhibitory phosphorylation of cdc2 on threonine 14 (Thr14) and tyrosine 15 (Tyr15) by Myt1 and Weel, respectively (9-11). Activation of this complex is triggered by the Cdc25C phosphatase through cdc2 dephosphorylation of Thr14 and Tyr 15 (12-14). In agreement with elevated cyclin A levels, KΔC-infected cultures did not significantly upregulate Cdc2 activity as evidenced by the high levels of Cdc2 tyrosine 15 phosphorylation (FIG. 4B). These results strongly suggest that KΔC induces a cell cycle block prior to mitotic entry, in G2. Supporting this, KΔC-positive cells were not found to assemble mitotic spindles 24 hours post-stimulation, at which point 60% of the cells displayed 4N DNA content (data not shown).

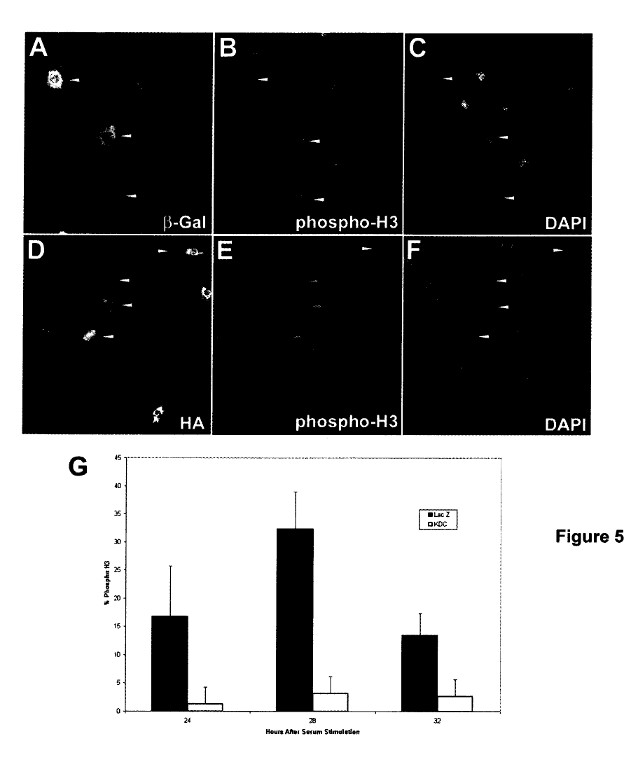

Chromosome condensation initiated in early G2 (38) is accompanied by the hyperphosphorylation of histone H1 (24) and phosphorylation of H3 (25) on serine 10 (Ser10) (26). To further refine the cell cycle block induced by KΔC expression, adenovirus-infected cultures were stained for both KΔC expression and phospho-H3. Double immunostaining of serum stimulated fibroblast cultures at 24 hours (FIG. 5AF) shows that H3 phosphorylation was markedly reduced in KΔC-expressing cells in comparison to control infected cultures. Although the proportion of HA- and phospho-H3-positive cells slightly increased at 28 and 32 hours post stimulation, their number was significantly lower than control cells, suggesting that KΔC-expressing cells are delayed in early G2.

### SLK Expression Induces Spindle Formation and Mitotic Entry

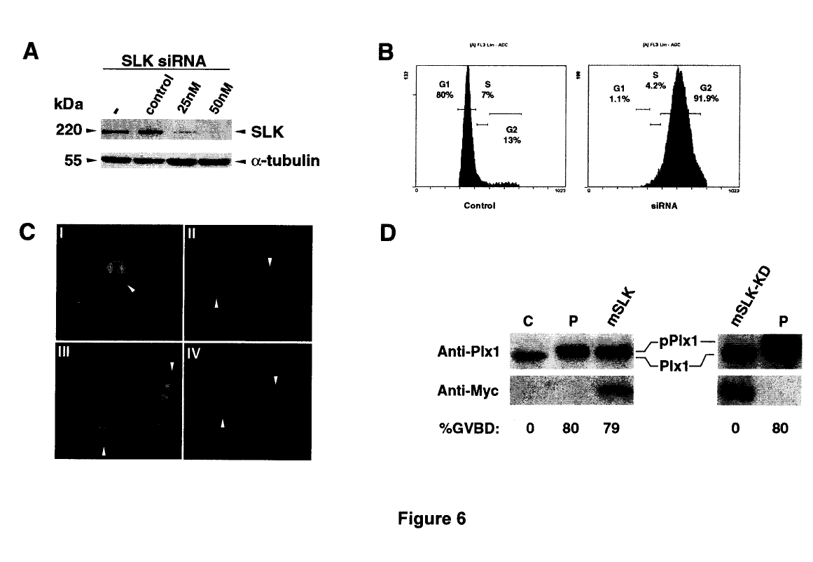

Our results show that expression of a kinase inactive SLK is sufficient to induce an early G2 cell cycle block in cycling fibroblasts, suggesting that an SLK-dependent pathway is required during G2 for progression into mitosis. To determine whether SLK plays a central role in G2 and to rule out potential non-specific effects by KΔC overexpression, exponentially growing fibroblasts were transfected with an SLK siRNA pool and subjected to DNA content analysis. Transfection of SLK siRNAs downregulated SLK protein levels by 80-90% within 48 hours (FIG. 6A). No effect was observed in the siRNA control samples. DNA content analysis 48 hours following siRNA transfection showed that SLK knockdown resulted in a marked G2/M accumulation (92% 4N DNA content; FIG. 6B), an inhibition of proliferation and increased cyclin A levels (data not shown). These data further support a role for SLK in cell cycle progression and rule out potential non-specific effects by KΔC overexpression. Supporting these results, microinjection of activated SLK (SLK1-373) in GFP-tubulin labeled cells, induced ectopic mitotic spindles in the injected cells within 3 to 6 hrs, ultimately resulting in cell death (FIG. 6C and (1)). Similarly, injection of the activated SLK, but not kinase dead, mRNA in Xenopus oocytes resulted in the hyperphosphorylation of Plx1 and germinal vesicle breakdown (GVBD) without progesterone induction, suggesting that the injected eggs re-entered the cell cycle as for progesterone-treated oocytes (FIG. 6D).

### Discussion

Cell cycle progression is regulated by complex signaling networks involving posttranslational modification, gene expression and cytoskeletal reorganization. Progress through the various phases involves the activation of key factors and is monitored by various “checkpoint” proteins. We have previously isolated a Ste20-like kinase termed SLK that is involved in cytoskeletal reorganization (1,30,31). Interestingly, a fraction of SLK protein is also observed to associate with the microtubule (MT) network in spreading and exponentially growing cells (1). Here it is shown that SLK also associates with the MT network at mitosis, suggesting that it plays a role in cell cycle progression. To investigate this, the activity and expression of SLK was evaluated throughout the cell cycle and the effect of a kinase-defective SLK on cell cycle progression was assessed.

The results show that SLK co-localizes with the mitotic spindle during M phase and that its kinase activity is upregulated as synchronized fibroblast cultures enter the G2/M compartment. Expression of an antisense SLK construct or a kinase-deficient mutant inhibited cell proliferation. Flow cytometric analysis of these populations showed that SLK is required for progression through the G2/M compartment and that cells expressing a kinase inactive SLK fail to downregulate cyclin A. Furthermore, we have observed that cultures expressing an inactive SLK show reduced histone H3 phosphorylation and p34Cdc2 activation. Supporting this, overexpression of activated SLK induced spindle formation and cell cycle re-entry in Xenopus oocytes. Overall our data suggests that SLK is required upstream of Cdc2 and that interfering with SLKdependent pathways leads to cell cycle arrest early in the G2 phase of the cell cycle.

Without wishing to be bound by theory or limiting in any manner, an interpretation of the data presented here indicates that SLK is required during G2, at a point after cyclin A expression and before Cdc2 activation, for progression into mitosis. A Xenopus Lok/Si tx10 homolog, xPolo-like kinase (xPlkk1), has been demonstrated to activate Plx1 (22). Interestingly, SLK has been demonstrated to phosphorylate and activate Plk in a mammalian system (33).

An evolutionary conserved relationship appears to exist between mammalian and amphibian Ste20 like kinases, the polo like kinase family, and the Cdc25C phosphatase. The human homolog of the Ste20 like kinase Lok, Stx10, has been shown to associate with and phosphorylate Plk1 (40) and Plk3 has been shown to initiate the nuclear translocation of Cdc25C by phosphorylating Cdc25C on serine 191(41). In Xenopus, the polo like kinase, Plx1, has been shown to activate Cdc25C (21), an event that has not been unarguably proven in a mammalian system and may be species specific. If Plk requires a signal from SLK to activate Cdc25C in a mammalian system, one would predict a G2/M arrest, similar to the findings presented here. This would result in a Cdc25C protein that is deficient in all of the post-translational modifications required to initiate mitosis. However, the questionable validity and specificity of phospho-specific Cdc25C antibodies has made the identification of the various forms of the phospho-Cdc25C difficult and inconclusive (not shown). We have previously shown that SLK can induce actin depolymerization and cell death in various cell lines (1,31). Without wishing to be bound by theory or limiting in any manner, one possibility is that SLK overexpression in cycling cells induces a deregulated mitotic entry, bypassing cell cycle controls, resulting in actin breakdown and death. Alternatively, SLK-mediated cytoskeletal reorganization may be required for, or trigger, checkpoint activation, G2 progression and mitotic entry.

### Example 2

### Implication of SLK in Modulating Cell Motility

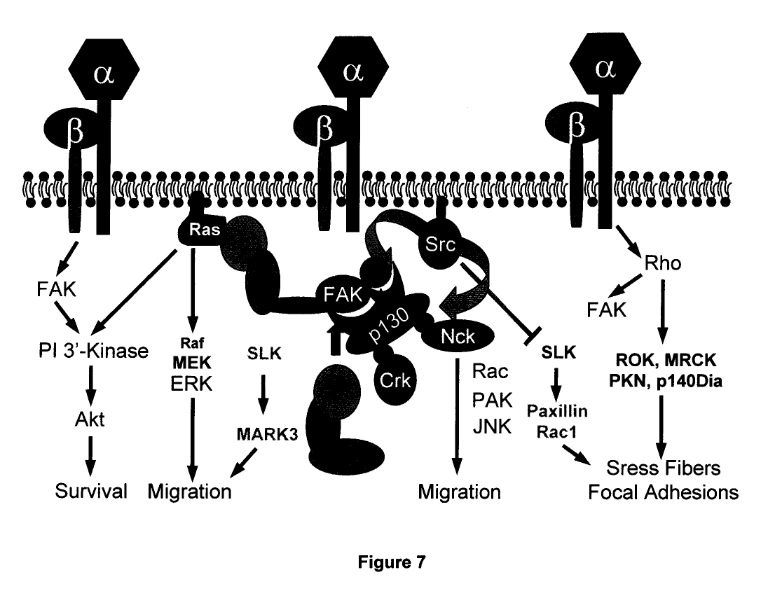

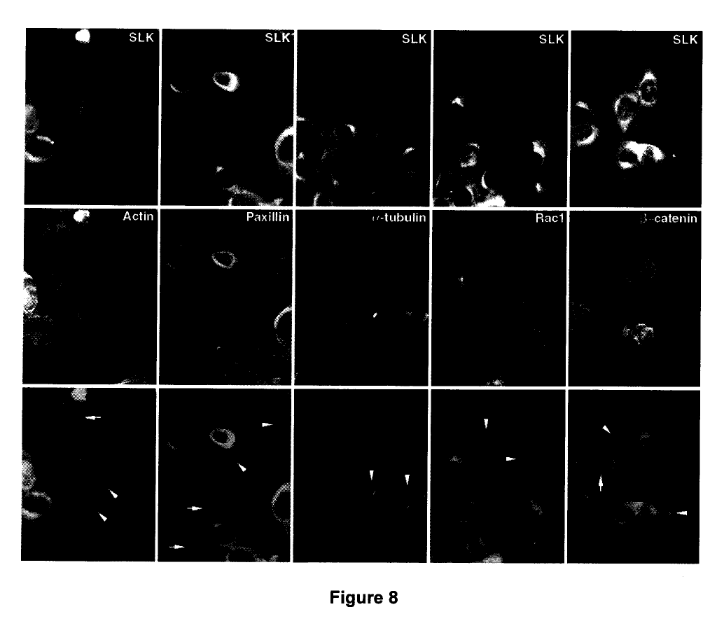

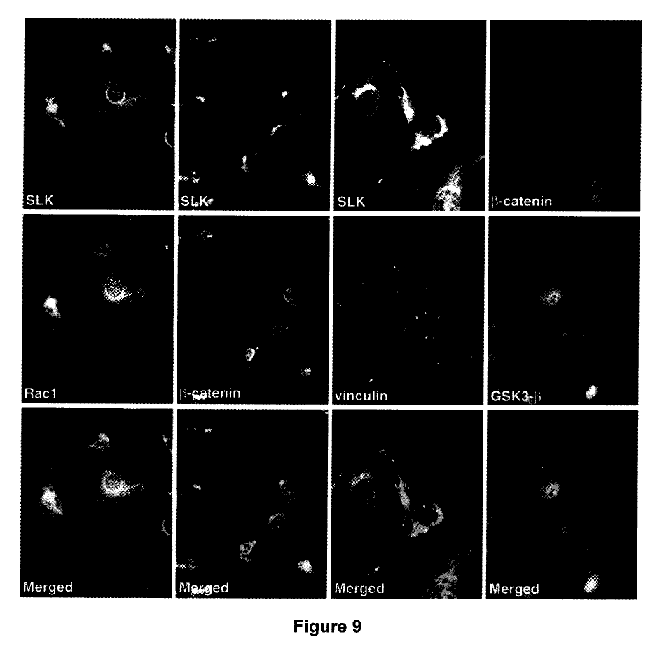

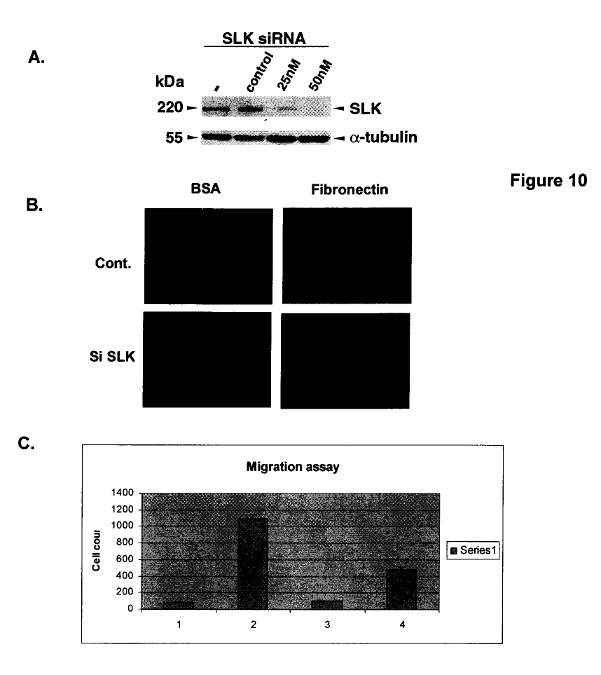

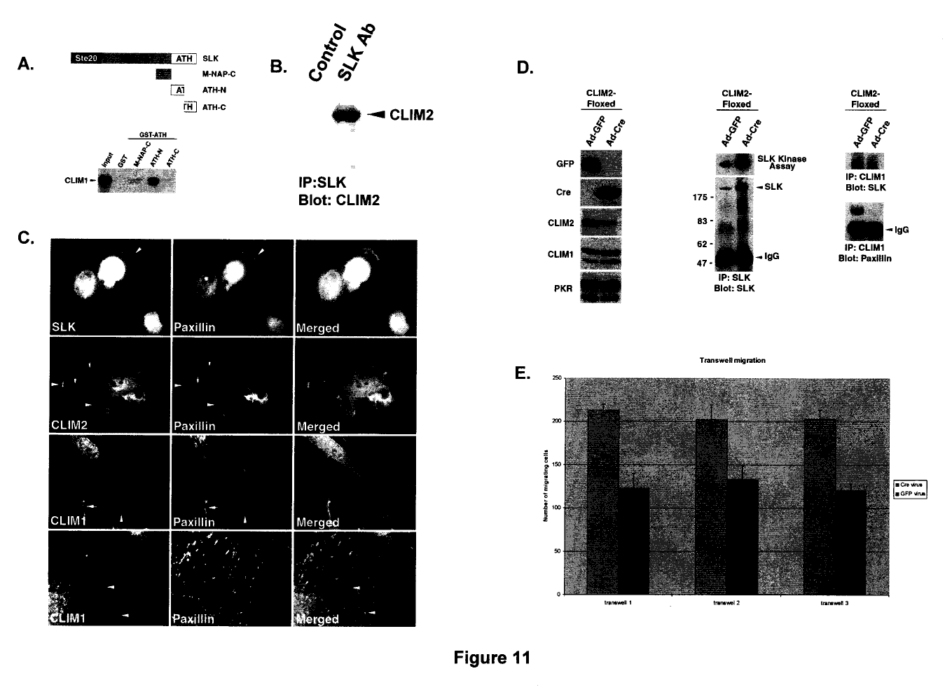

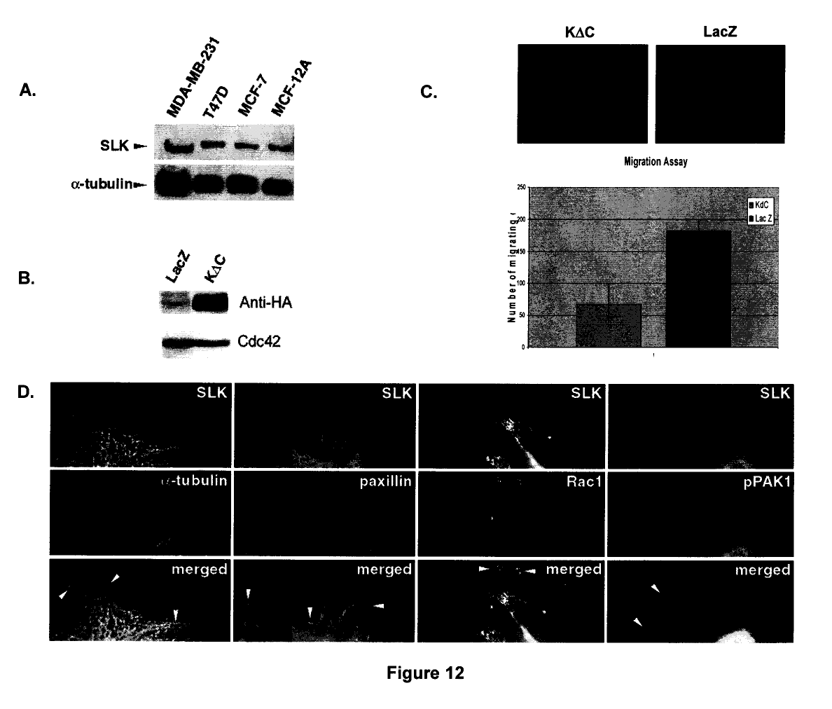

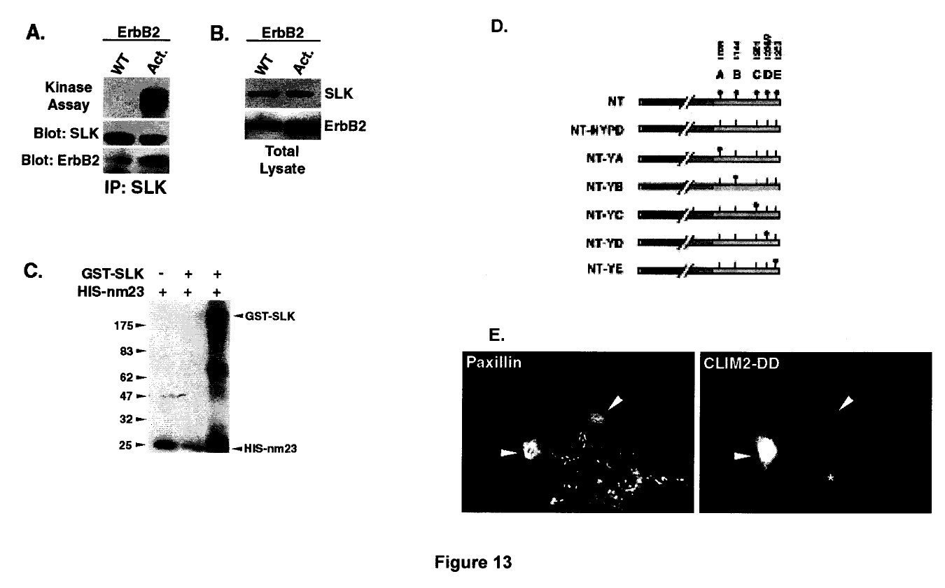

Without wishing to be limiting or bound by theory, FIG. 7 shows a diagrammatic representation of integrin-stimulated signaling events involving FAX. The role of SLK in cell growth and migration was investigated. In particular, our results suggest a role for SLK and one of its binding partners, CLIM2, in cell motility. Wound healing of fibroblast monolayers induces the recruitment of SLK to the leading edge of migrating cells with Rac1, GSK-3b, paxillin and the microtubule network (FIGS. 8-9). In addition, expression of SLK dominant negative mutants or siRNAs inhibit cell motility by 60-70% in wound healing and Boyden chamber migration assays (FIGS. 10, 12). Interestingly, deletion of CLIM2 through Cre-mediated recombination, increases cell motility on fibronectin matrix, and SLK antibody accessibility, suggesting that CLIM2 may regulate SLK activity through conformational change (FIG. 11). To gain a better understanding of the role of SLK during cell motility, we have assayed its subcellular localization and kinase activity following the stimulation of cultured cells with various growth factors known to induce a migratory response. Of interest was the finding that stimulation of MCF-7 cells with heregulin b1 (HRG) induced lamellipodia formation, migration and recruitment of SLK, phospho-PAK1 and Rac1 to the leading edge (FIG. 12), suggesting that ErbB2-mediated migration may implicate SLK. Supporting this, co-expression of wildtype Myc-SLK and Neu-NT (activated rat ErbB2) in fibroblasts resulted in a marked increase in SLK kinase activity, suggesting that ErbB2 signaling activates SLK. Furthermore, immunoblotting of the SLK immunoprecipitates with anti-ErbB2 shows that Neu-NT and SLK can co-precipitate, suggesting that SLK can be recruited to the activated ErbB2 receptor complex (FIG. 13).

We have identified several SLK binding proteins, including the CLIM2 adapter protein (FIG. 11). In addition, we have also isolated the antimetastatic gene nm23 (nucleoside diphosphate kinase; NDPK) as an SLK regulatory protein (FIG. 13). Interestingly, the ErbB2 status in various cell lines has been inversely correlated with the levels of nm23 expression, suggesting that the increased invasiveness of ErbB2 overexpressing cells implicates the loss of nm23 expression. We have observed that nm23 is not a substrate for SLK but rather inhibits its activity in vitro through direct interaction (FIG. 13). Without wishing to be bound by theory, this type of regulation may be lost in ErB2-positive tumors, leading to higher SLK activity and increased motility and invasion.

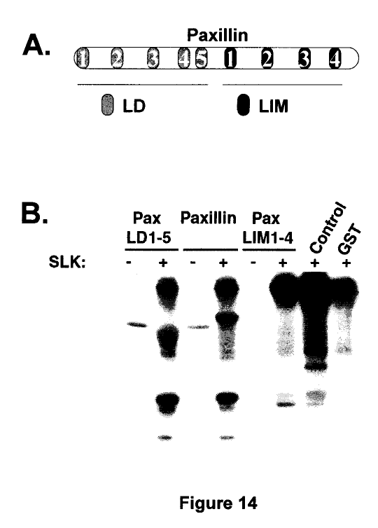

In addition to the regulation of nm23 expression, one study has shown that HRG stimulation of MCF-7 results in the serine phosphorylation of the signaling adapter paxillin. Serine phosphorylation of paxillin is associated with increased adhesion turnover and motility. We have recently found that SLK can phosphorylate paxillin in vitro in its LD domains (FIG. 14). Without wishing to be bound by theory, one possibility is that ErbB2 activation and SLK stimulation may play a role in adhesion dynamics and cell motility through the regulation of adapter protein function.

### Example 3

### Lbd2 and Lbd1 Regulation of SLK

### Cell Transfections and Cell Culture

Ldb1-floxed primary MEFs were generated from homozygous Ldb1 floxed 13.5 dpc embryos. Briefly, the internal organs were removed and the embryos were minced 3 ml of trypsin-EDTA solution (0.05%, Gibco). Dissociated embryos were incubated for 3 minutes at 37° C. followed by the addition of complete growth medium. The tissue was then triturated several times and incubated at 37° C. with 5% CO₂ for 1-3 days. After 3-4 days in culture the cells were split 1:4, grown for 48 h and frozen as live stocks. All experiments were performed on cultures passaged 2-6 times. MEF 3T3, NIH3T3 and primary MEFs were all maintained in Dulbecco's modified MEM (DMEM, Gibco) supplemented with 10% fetal bovine serum (FBS, Gibco), 2 mM L-glutamine (Gibco) and penicillin G (200 U/ml, Gibco) and streptomycin sulfate (200 μg/ml, Gibco) in a humidified 37° C. incubator at 5% CO₂. DNA transfections into cultured cells were performed using lipofectamine and plus reagent (Invitrogen) according to manufacturers recommendations using a total of 2 μg of plasmid DNA. Rat primary cortical neurons were generated essentially as described (60). Embryos were obtained from pregnant Sprague-Dawley rats at embryonic day 15 and the cortical tissue removed to 1× Hank's balanced salt solution (HBSS). Tissue was triturated several times, incubated with trypsin and DNase for 30 minutes at 37° C. with shaking. Cells were recovered and plated on poly-L-lysine coated dishes in neurobasal medium supplemented with B2 and N27.

### Boyden Chamber Migration Assays and Scratch Wound Migration Induction

MEF 3T3 or NIH 3T3 cells were either co-transfected as described with GFP and deletions of Ldb1 and 2, treated with siRNA to either SLK, Ldb1, 2 or both Ldb1 and 2 and serum starved overnight. Cells were trypsinized the following day and trypsinization halted with the addition of soybean trypsin inhibitor (1×, Sigma). Cells (1−3×10⁴) were resuspended in DMEM containing 0.5% BSA and added to the top of a Boyden transwell migration chamber pre-coated with fibronectin (10 μg/ml). The cells were then allowed to migrate for 3 hours. Residual cells were removed from the top of the chamber and the filter was rinsed in PBS, fixed in 4% PFA for 10 minutes and stained with DAPI (0.5 mg/ml, Sigma). The cells that migrated to the underside of the filter were enumerated using DAPI fluorescence. 5 to 10 random fields were counted. Cell counts were performed in triplicate for three independent experiments. Representative experiments are shown. Scratch wound induced migration was performed as described (52). Briefly, MEFs were plated on fibronectin (10 μg/ml) and confluent monolayers were then scratched with pipette tips such that 50% of the monolayer was removed. Cells were then washed with PBS, refed and collected at various time points.

### SiRNA Knockdown of SLK, Ldb2 and Ldb1

NIH 3T3 cells plated at a density of 3×10⁵ in 60 mm plates were transfected with 50 or 100 nM siRNA (Dharmacon) duplex for SLK (5′-GGUUGAGAUUGACAUAUUA) (SEQ ID NO:6), Ldb2 (5′-ACAAGCAGCACGUCCAAUAUU) (SEQ ID NO:7) or Ldb1 (5′-GAACUUAUGUCCCGCCACAUU) (SEQ ID NO:8) using the Trans-IT TKO transfection reagent (Mirus Corp.) according to manufacturers recommendations. Cells were collected at 24 or 48 hours post-transfection and assayed for cell migration by Boyden chamber and protein expression by western blot analysis. Control siRNAs consisted of Dharmacon's non-targeting duplex. Similar results were obtained with scrambled siRNAs.

### Plasmid Constructs,

DNA plasmids were constructed using standard molecular cloning techniques. Myc-tagged SLK plasmids were constructed as described (31). The GST-SLK plasmids were generated by subcloning the corresponding fragments from the Myc-tagged versions (Sabourin et al., 2000) to pGEX-5× or 4T1 (Amersham). The yeast two hybrid SLK-ATH bait plasmid was constructed by inserting the ATH (XhoI digested, blunted with Klenow) domain of SLK in frame with the Gal4 DNA binding domain in pAS2 (BD Clontech). Ldb1 and 2 were excised from pACT2 (XhoI/EcoRI) and subcloned in frame into Myc and HA epitope-tagged vectors (pCAN-HA or pCAN-Myc). The following Ldb2 constructs were generated by excising fragments of Ldb2 from HA-Ldb2 and recloning into pCAN-HA. HA-Ldb2 DLBD (aa 1-296), HA-Ldb2 DD (aa 1-186), HA-Ldb2 NLS (aa 188-288), and HA-Ldb2 DDDC (aa 1-124). HA-Ldb2 LBD (aa 298-373) was generated by PCR (5′-ggggggatccagctgcaaacctgagtctgtcc-3′ (SEQ ID NO:9) and 5′-gggggaattcacgggcctattgacagtggattct-3′) (SEQ ID NO:10) using VENT polymerase (NEB). All clones were verified by DNA sequencing.

### Antibodies and Immunofluorescence

The primary antibodies used in these studies were as follows: SLK polyclonal antibodies were as described previously (31), Ldb1 (Santa Cruz), Paxillin (BD Transduction labs), PKR (Santa Cruz), α-tubulin (Sigma) and Ldb2 (Abcam) was used at 1:100.

For immunofluorescence studies, mouse embryo fibroblasts were plated on coverslips coated with or fibronectin (10 μg/ml) and incubated overnight. The following day cells were rinsed with PBS, fixed in 4% PFA and blocked in PBS containing 5% goat or donkey serum and 0.3% triton-X100 for 20 minutes. Fresh blocking solution containing primary antibody was added and incubated for 1 h at room temperature. Antibodies were detected with either anti-mouse, anti-goat or anti-rabbit secondaries conjugated to either fluorescein isothiocyanate (FITC) or tetramethyl rhodamine isothiocyanate (TRITC) (Sigma). The samples were visualized with a Zeiss Axioscope100 epifluorescence microscope equipped with the appropriate filters and photographed with a digital camera (Sony Corporation HB050) using the Northern Eclipse software package.

### Western Blotting and Immunoprecipitation and Kinase Assays

Cells were lysed in RIPA buffer as previously described (44) and lysates were cleared by centrifugation at 10000 g for 2 minutes. Protein concentrations were determined using protein assay dye reagent (Biorad). Equal amounts of protein (20-40 μg) were electrophoresed on 8-15% polyacrylamide gels and transferred to PVDF membrane. Membranes were probed with the indicated antibodies overnight at 4° C. in 5% skim milk powder in 1×TBST (50 mM Tris pH 7.4, 150 mM NaCl, 0.05 Tween 20). Membranes were washed in TBST and incubated with horseradish peroxidase coupled secondary antibodies and the reactive proteins were detected using chemiluminescence (Perkin Elmer) and exposure to X-ray film.

For immunoprecipitations, 300 μg of protein lysate was immunoprecipitated with 2-3 μg of antibody and 20 μl of protein A sepharose (Pharmacia) for 2-12 hours. Immunecomplexes were recovered by centrifugation and washed with NETN buffer (20 mM Tris-HCl pH 8.0, 1 mM EDTA, 150 mM NaCl, 0.5% Nonidet P-40) and subjected to SDS-polyacrylamide gel electrophoresis (PAGE) or kinase assay.

In vitro SLK kinase assays were performed following SLK immunoprecipitation as described previously (44). Kinase reactions were stopped by adding 7 ml of 4× sodium dodecyl sulfate (SDS) sample buffer and electrophoresed on 8% SDS-PAGE. The gels were transferred to PVDF membranes and subjected to autoradiography followed by western blotting with SLK antibody.

### In Vitro Binding Assays

GST fusion proteins were generated by induction of bacterial cultures with 1 mM isopropyl-beta-D-thiogalactopyranoside (IPTG, Sigma) for 2 hours. Bacteria were pelleted, resuspended in 1 ml bacterial lysis buffer (20% sucrose, 10% glycerol, 50 mM Tris-HCl pH 8.0, 2 mM MgCl₂, 2 mM DTT, 10 μg/ml leupeptin, 10 μg/ml pepstatin, 10 μg/ml aprotinin, 1 mM phenylmethylsulphonylfluoride [PMSF] and 100 μM benzamidine) and sonicated on ice.

Glutathione sepharose beads (Amersham Pharmacia) were added to the cleared supernatants and bound GST fusions were collected by centrifugation, washed three times with NETN and subjected to binding assays. In vitro translated proteins were generated using the TNT quick coupled in vitro transcription translation kit (Promega) according to the manufacturer's instructions. Translated proteins were incubated with either GST or GST fusions in NETN buffer, washed with NETN and eluted from the beads by boiling in sample buffer. Proteins were fractionated by SDS-PAGE, the gels were stained with Coomassie brilliant blue (Sigma) to visualize the proteins, destained (30% methanol, 10% acetic acid), dried and subjected to autoradiography.

### Recombinant SLK Kinase Assay

Recombinant SLK was prepared by inducing an overnight culture of GST-SLK with IPTG and preparing GST SLK as described above. Recombinant His-tagged Ldb2 and 2 were prepared by inducing overnight cultures with IPTG and purifying recombinant protein through a Ni-NTA columns (Qiagen) according to manufacturer's instructions. Kinase assays were performed as described above.

### Yeast Two-Hybrid Analysis

Yeast two-hybrid screens were performed as suggested by the manufacturer (BD Clontech). Briefly, the ATH domain of SLK was subcloned into pAS2, in frame with the GAL4-DBD as described above. Yeast strain AH109 (BD Clontech) was transformed and tested for self activation using the endogenous LacZ reporter and His auxotrophy. The resulting AH109 clones were mated with library (mouse E10.5 cDNA) pre-transformed Y187 and His/Trp/Leu auxotrophs were screened for positive interaction. Of the resulting 29 clones, 5 contained the Ldb1 cDNA and three the Ldb2 cDNA. cDNAs were subcloned into HA or Myc-tagged vectors and further tested for ATH binding.

### Results

### SLK is Activated by Monolayer Wounding and is Required for Cell Migration

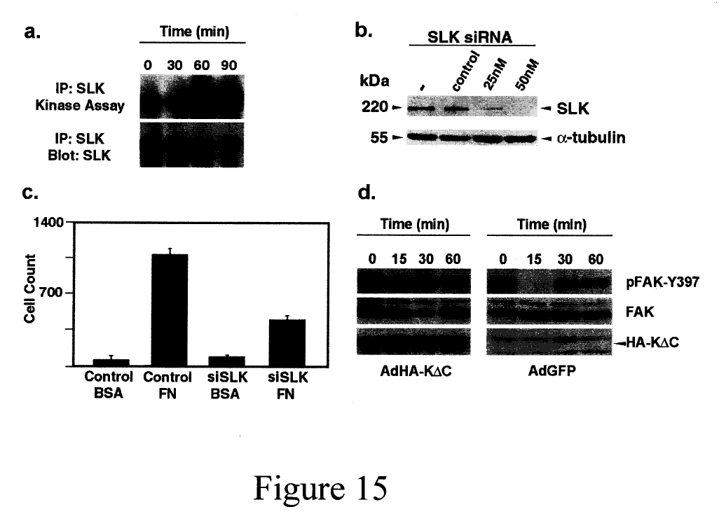

We have previously shown that SLK can be co-precipitated with α-tubulin and that it localizes to membrane ruffles at the periphery of spreading fibroblasts (1). In addition, SLK induces cytoskeletal rearrangements through a Rac1-mediated pathway (1). Therefore, we hypothesized that SLK plays a role in cell migration and investigated its activity at various time points following scratch wound induced migration of fibroblast monolayers (52). In vitro kinase assays show that SLK activity markedly increased following scratch wounding of confluent fibroblasts, reaching a maximum at about 60 minutes, suggesting a role for this kinase in cell migration (FIG. 15a). To further investigate the role of SLK in cell migration, we transfected short interfering RNA (siRNA) molecules specific for SLK into MEF-3T3 fibroblasts and assayed migration of these cells in a transwell assay. Levels of SLK protein were efficiently reduced at 25 nM of siRNA and undetectable at 50 nM compared with control siRNA treated cells (FIG. 15b). Cells treated with SLK siRNA showed a 50-60% decrease in migration compared with cells treated with control siRNA (FIG. 15c). These data strongly support a role for SLK in the process of cell migration. To gain mechanistic insights into SLK function, MEF-3T3 cells were infected with adenovirus carrying a dominant negative SLK (SLK1-373K63R; ATP-binding site mutant) or a GFP control. Infected cultures were subjected to microtubule-dependent focal adhesion turnover assays and surveyed for FAK-pTyr397 levels (53). As shown in FIG. 15d, a marked delay in FAK-pTyr397 reduction was observed in SLK1-373K63R expressing cultures following nocodazole wash-out, suggesting that SLK-dependent signals are required to mediate focal adhesion turnover.

Ldb1 and 2 Interact with SLK ATH Domain

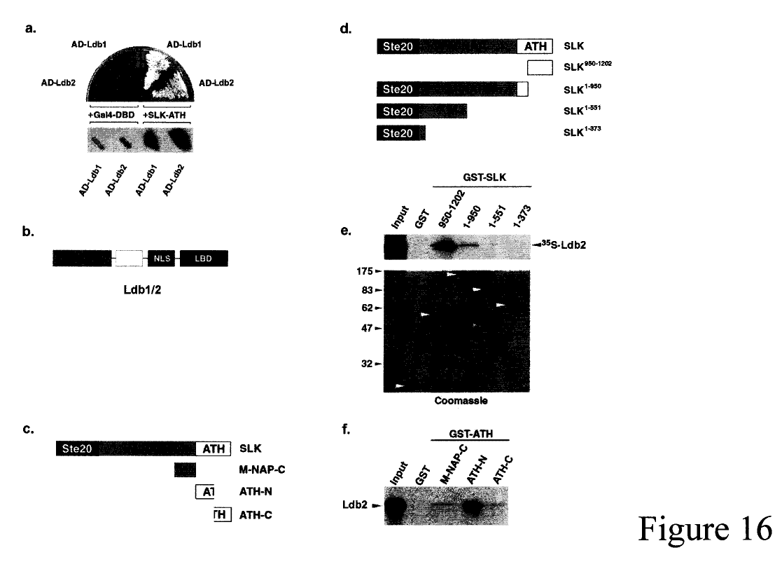

Our previous studies have shown that deletion of the SLK ATH domain increases its kinase activity, suggesting that it plays a role in SLK regulation (31). To gain further insight into the autoinhibitory role of the ATH domain, we performed a yeast two-hybrid screen using a GAL4-DBD-ATH domain fusion as bait. Surprisingly, we identified the transcription co-factors Ldb1 and Ldb2 as ATH-interacting proteins (FIG. 16a). Ldb factors [reviewed in 54] contain an amino terminal dimerization domain, a carboxy-terminal LIM binding domain and a central domain containing a nuclear localization signal (see FIG. 16b). We sought to confirm the interaction of Ldb1 and 2 with the SLK-ATH domain in yeast. Ldb1 and 2 interacted with the ATH-GAL4-DBD fusion but not the GAL4-DBD, indicating that the Ldb factors associate specifically with the ATH domain (FIGS. 16a and b). Similarly, patching of these clones in a β-galactosidase reporter assay resulted in the activation of the LacZ reporter gene (FIG. 16a).

Interactions of Ldbs and SLK were confirmed by direct binding assays. In vitro translated Ldbs labeled with [35S]-methionine was incubated with various GST-SLK fusion protein deletions (FIGS. 16c and d). Following pulldowns, Ldb2 had the highest affinity for the SLK ATH domain (SLK 950-1202, FIG. 16e) and moderate affinity for a carboxy-terminal deletion of SLK lacking the latter two thirds of the ATH domain (SLK 1-950). Prolonged exposures also revealed weak binding to deletions of SLK lacking the ATH domain (GST-SLK-1-551, GST-SLK-1-373). No binding was observed to GST alone (FIGS. 16e and f). Ldb1 was also able to bind directly to the SLK-ATH domain in vitro in a manner that was indistinguishable from Ldb2 (not shown). Together, these data indicate that Ldb1 and 2 preferentially target the ATH domain of SLK in vitro.

To refine the binding region of SLK to Ldb2. GST fusions of the of the SLK MNAP carboxy-terminus and the ATH amino and carboxy-termini were assayed for binding to [35S]-labeled Ldb2 (FIGS. 16e and f). Binding of Ldb2 to the MNAP carboxy-terminus and the ATH carboxy-terminus was minimal. Strong binding to a 109 amino acid stretch of the ATH amino-terminus was observed indicating that Ldb2 binds this region of SLK with highest affinity (FIG. 16f).

### Determination of SLK Interacting Regions of Ldb2

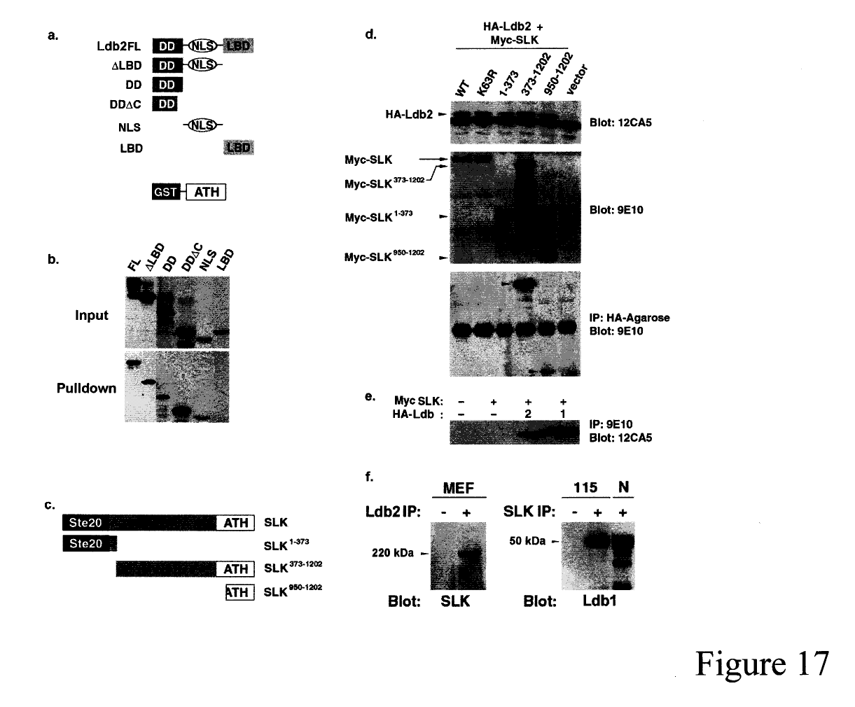

To map the Ldb1/2 domains of interaction, [35S]-labeled deletions of Ldb1 were assayed for binding to a GST-tagged SLK ATH domain (FIG. 17a). Interestingly, a 186 amino acid portion of the dimerization domain (DD) and a 100 amino acid fragment of the nuclear localization signal (NLS) region of Ldb2 could independently bind the SLK ATH domain in vitro (FIG. 17b). In addition, versions of Ldb2 lacking the C-terminal LIM binding domain (ΔLBD) were able to bind the SLK ATH domain while the LBD of Ldb2 by itself could not bind (FIG. 17b). Further deletion of 62 amino acids from the carboxy terminal end of the dimerization domain (DDΔC) retained binding thus refining the Ldb2 binding region to the SLK ATH domain in the C-terminal 124 amino acids of Ldb2. An analogous set of Ldb1 deletion mutants were tested for binding to GST-ATH with similar results (data not shown). Therefore, Ldb2 and 2 likely contact the amino terminal half of the SLK ATH domain through both their dimerization and NLS domains.

Association of Ldb2 and 2 with SLK In Vivo

We next tested whether Ldb1 and 2 were able to bind the SLK ATH domain in cultured cells. To address this, HA-tagged Ldb2 and Myc-tagged SLK deletion constructs (FIG. 17c) were co-transfected into 293 human embryonic kidney cells and subjected to immunoprecipitation and western blot analysis (FIG. 17d). Following co-immunoprecipitation analyses, Myc-tagged SLK deletion constructs containing the ATH domain (Myc SLK 373-1202 and Myc SLK 950-1202) were found to co-precipitate with HA-Ldb2. Similarly, HA-Ldb1 could also be co-precipitated with these SLK truncations (not shown). Surprisingly, full length SLK and kinase inactive (Myc-SLK, Myc-SLK K63R) did not co-immunoprecipitate with HA-Ldb2. This suggests that the presence of the SLK kinase domain either masks the interaction between the ATH domain and Ldb2 or alternatively renders the epitope tag on Ldb2 inaccessible to the antibody. Supporting the latter, reciprocal immunoprecipitations showed that Myc-tagged Ldb2 could be detected in HA-SLK immune complexes (FIG. 17e). Similarly, Myc-SLK 373-1202 and Myc-SLK 950-1202 also co-precipitated with HA-tagged Ldb1 (data not shown). Taken together, these experiments confirm that the ATH domain of SLK is required for interaction with Ldb1 and 2 in vivo. Without wishing to be bound by theory, this data suggests that SLK adopts a conformation whereby the kinase domain either interferes with ATH/Ldb association or blocks access to epitopes on the Ldb amino-terminus. Endogenous Ldb1 was also found to co-precipitate with SLK in IP-western blot analyses using lysates from fibroblasts, neuroblastoma cells and primary cortical neurons (FIG. 17h).

Ldb Proteins Co-Localize with SLK at Cytoskeletal Structures

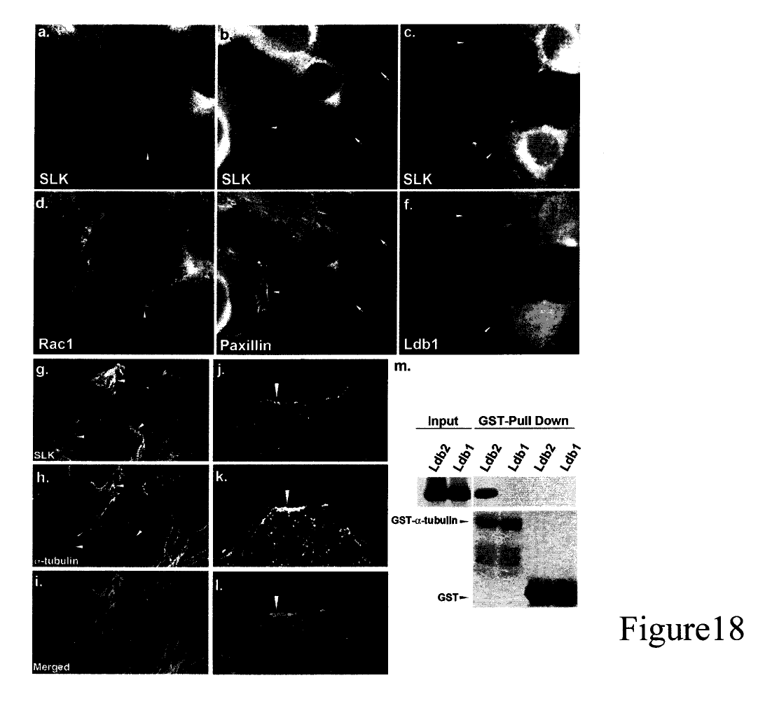

We have established that Ldb factors associate with the ATH domain of SLK in vitro and in living cells. We then sought to investigate whether the SLK/Ldb complex also co-localized in migrating cells. We first performed immunostaining of endogenous Ldb1 and SLK in C2C12 myoblasts (not shown) and mouse embryo fibroblasts (MEFs) plated on collagen and fibronectin respectively (FIG. 18a-f). Double immunofluorescence studies showed that the two proteins co-localized at the leading edge of migrating cells in membrane ruffles. Similarly, SLK can be co-localized with paxillin and Rac1 in membrane ruffles (FIG. 18a-f).

We have previously shown that SLK is indirectly associated with microtubules in spreading and migrating cells (1). High magnification of leading edge immunostains shows that SLK, Ldb2 and Ldb1 (not shown) co-localize with α-tubulin in membrane ruffles. Since SLK does not bind tubulin directly, we tested the possibility that it may be tethered to the microtubule through Ldb2 or Ldb1. Supporting our immunofluorescence results, Ldb2, but not Ldb1, was found to bind α-tubulin in vitro, indicating that Ldb2 may act as a link between SLK and the microtubule network. Taken together, these data indicate that SLK/Ldb complexes exist on microtubules and at the leading edge of migrating cells, further supporting a role for this complex in cell motility.

### Ldbs Regulate SLK Structure and Activity

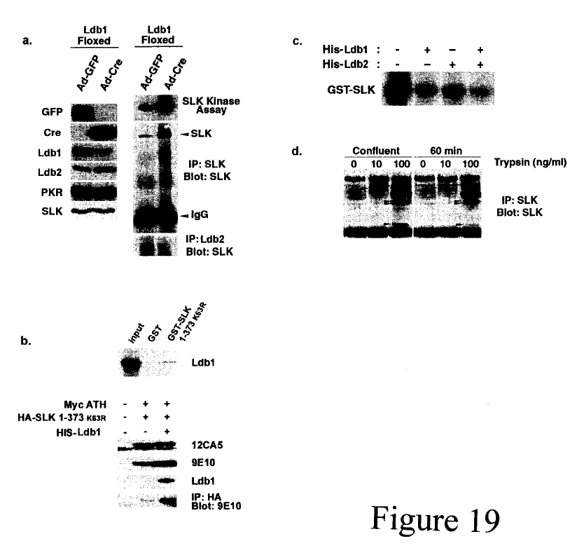

To assess the role of Ldb1 in SLK function, we used MEFs derived from mice homozygous for a ‘floxed’ Ldb1 locus (FIG. 19a). While Ldb2 and SLK levels remain unchanged, western blot analysis of extracts from MEFs infected with adenoviral vectors carrying GFP or Cre recombinase showed a Cre-mediated reduction in Ldb1 levels (FIG. 19a). We then investigated SLK activity and association with endogenous Ldb2 in the absence of Ldb1. Although no change in SLK activity was observed after normalization, Cre-mediated reduction of Ldb1 levels consistently resulted in a marked increase in SLK immunoprecipitation efficiency compared to GFP-infected cells (FIG. 19a). This suggests that disruption of the SLK/Ldb1 complex exposes additional epitopes on SLK. Surprisingly, reduced Ldb1 levels also disrupts SLK association with Ldb2, indicating that Ldb1 is required for the formation of a complex containing Ldb2 and SLK. (FIG. 19a). Without wishing to be limiting or bound by theory, we hypothesized that the SLK/Ldb interaction may result in an SLK inhibitory conformation important for its regulation. Supporting this, weak binding of Ldb1 to the kinase domain (GST-SLK1-373) can be observed (FIG. 19b). Interestingly, a stronger in vivo interaction between the kinase domain and the ATH was observed when Ldb1 was co-expressed (FIG. 19b), indicating that Ldb1 can mediate and perhaps stabilize the folding of the kinase region onto the ATH domain. We reasoned that this tripartite complex would result in altered SLK kinase activity. To investigate this, we performed in vitro kinase assays with GST-tagged recombinant SLK alone or in the presence of recombinant His-tagged Ldb1 and 2 (FIG. 19c). Our results indicate that addition of either Ldb1 or 2 results in reduced SLK activity and this effect is most pronounced when both Ldb1 and 2 are present together (FIG. 19c), supporting the notion that Ldb factors regulate SLK activity through direct interaction. Supporting this, differential banding patterns were observed when SLK immune complexes from migrating cells were subjected to protease access assays (FIG. 19d). This suggests that the activation of SLK during cell migration is accompanied by a conformational change.

### Increased Cell Migration in Ldb-Deficient Cells

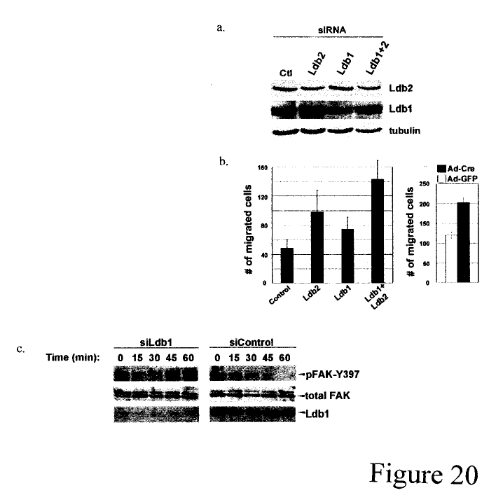

The observation that Ldb factors regulate SLK activity led to the hypothesis that downregulation of Ldb1 or 2 may result in an SLK “open” conformation and increased motility. We therefore transfected Ldb1/2 siRNAs in NIH 3T3 cells and assayed migration rates using Boyden chambers. Following siRNA treatment, the levels of Ldb2 and 2 protein were reduced by at least 50% compared to siRNA control (FIG. 20a). Reducing Ldb1 protein resulted in a 1.5 fold increase in migration while reduction of Ldb2 enhanced migration two-fold compared with siRNA control transfected cells (FIG. 20b). Reduction of both Ldb1 and 2 resulted in almost a four-fold enhancement of migration (FIG. 20b). Consistent with the siRNA data, Cre-mediated deletion of Ldb1 in MEFs resulted in approximately a 1.5 fold increase in migration compared to control infected cells (FIG. 20b). Supporting these observations, knock down of Ldb1 in NIH3T3 cells resulted in faster focal adhesion turnover in nocodazole wash out experiments (FIG. 20c). Whereas Ldb1 siRNA treated cells re-assembled focal adhesions about 45 minutes post-wash out, control-treated cells did not show re-assembly for up to 60 minutes. Taken together, these data suggest that in the absence of Ldb1, SLK conformation is altered and this correlates with enhanced migration.

### Discussion

SLK and cell Migration

We have previously observed that SLK can indirectly associate with the microtubule network and mediate actin stress fibre dissolution in a Rac1-dependent manner 3.

Here, we have shown that interfering with SLK levels or activity negatively affects cell migration and microtubule-dependent focal adhesion turnover, suggesting that SLK is required for cell motility. Without wishing to be limiting or bound by theory, one possibility is that SLK is an actin disassembling-factor required for the destabilization of focal adhesions/contacts during migration. It has been reported that microtubules target focal contacts to modify their characteristics, including their disassembly (55, 56). Therefore, SLK may represent a microtubule-associated signal required to induce actin and adhesion remodeling during migration.

Ldb1 and 2 Association with SLK

Our results show that Ldb1 and 2 associate with the ATH domain of SLK in vitro and in vivo. Both the dimerization and NLS domains of Ldb1 and 2 independently bind the SLK ATH domain in vitro, suggesting a strong multi-contact association between these proteins. Since Cre-mediated depletion of Ldb1 did not affect the intracellular distribution of SLK (not shown) we propose that the association of Ldb1 and 2 with SLK likely controls its activity as the kinase activity of recombinant SLK is drastically reduced when incubated with either Ldb1 or 2 (see FIG. 19c). Without wishing to be bound by theory, the mechanism by which these factors inhibit SLK activity likely involves SLK conformational changes imparted by Ldb1/2 binding. It has been recently suggested that SLK can form homodimers (Ref 57 and Storbeck unpublished). Supporting this, yeast two-hybrid screens with full-length kinase inactive SLK retrieved a number of independent clones all encoding the MNAP region of SLK suggesting that it may self-associate through this domain (Wagner, unpublished results). In the case of LIM homeodomain transcription factors, a Ldb1 homodimer is required to link two of these proteins together in a functional complex (51, 57). Without wishing to be bound by theory, we propose that a Ldb1/2 heterodimer can form a tetrameric complex with two SLK molecules. Evidence for heterodimerization of these factors includes the fact that Ldb2 association with SLK is lost upon removal of Ldb1 following adenoviral Cre infection in ‘floxed’ Ldb1 MEFs and that tagged versions of Ldb1 and 2 can be co-immunoprecipitated. It is then possible that Ldb association inhibits SLK activity by sequestering the kinase domain in a complex with the SLK ATH domain and the Ldb dimerization domain (FIG. 20). This structural arrangement is suggested by the observation that the Ldb proteins can enhance the formation of a tripartite association between Ldb/ATH and the kinase domain when co-expressed as individual domains (see FIG. 19). Combined with Ldb1 deletion, resulting in enhanced recovery of active SLK by immunoprecipitation, protease access assays suggest that Ldb1/2 association maintains SLK in a restrictive conformation. This conformational change upon activation may allow SLK greater access to substrates. Therefore, SLK dimers may adopt a low activity conformation with a Ldb1/2 heterodimer. A somewhat analogous structure is adopted by PAK, also a Ste-20 kinase family member. PAK homodimers are maintained in a folded inactive conformation by the binding of the amino-terminal regulatory domain of one PAK molecule to the kinase domain of the other (58). Interestingly, for both PAK2 and SLK, the regulatory domain can be cleaved by caspases during apoptosis releasing truncated unregulated kinase domains which potentiate cell death (31, 59). Our data show that SLK, paxillin and Ldb1 can all be co-localized to ruffles in migrating fibroblasts. Furthermore, a proportion of SLK and Ldb2 has been found to decorate the microtubule network. Furthermore, we find that Ldb2 can bind α-tubulin directly. One possibility is that this complex is recruited to focal contacts at the leading edge where it modulates actin stability and focal complex dynamics through the phosphorylation of SLK target proteins. Supporting this, expression of dominant negative forms of SLK in fibroblasts resulted in reduced migration rates and delayed microtubule-dependent adhesion turnover, whereas Ldb1 deletion increased it. Overall, our data suggest that SLK is a novel regulator of cell migration and that Ldb1/2 heterodimers can bind directly to its ATH domain, a site of important negative regulation (FIG. 20). During cell migration modification of this complex may result in a SLK conformational change, increasing its activity, focal contact turnover and migration rate.

### Example 4

ErbB-2/HER2 is a member of the epidermal growth factor (EGF) receptor tyrosine kinase (RTK) family. It has been shown that about 25-30% of human breast cancers overexpress ErbB2 and present with poor prognosis due to more invasive tumors. Despite the importance of HER2/ErbB2 in the etiology of breast carcinomas, the molecular mechanisms by which activated ErbB2 confers an invasive phenotype is still unclear.

As discussed herein, cell migration involves a multitude of signals converging on cytoskeletal reorganization, essential for development, immune responses and tissue repair. More importantly, increasing evidence show that the cell migration machinery is required for invasion and metastasis of tumor cells. Here we show that the microtubule-associated Ste20 kinase SLK is required for cell migration and is activated by scratch wounding of cell monolayers. SLK activation requires the src-family kinases and Focal adhesion kinase. Knockdown of SLK in fibroblasts and breast carcinoma inhibits cell motility. Interestingly, we find that SLK is activated in HER2/ErbB2 overexpressing cells and Heregulin treatment in breast carcinoma. Significantly, interfering with SLK levels or activity in breast carcinoma results in a block of Heregulin-induced chemotaxis in vitro. We have also shown that SLK is regulated by the metastasis suppressor nm23, a gene frequently downregulated in invasive tumors and HER2-positive carcinomas. We show that nm23 inhibits SLK activity and that this direct binding is lost upon activation by Heregulin treatment. The SLK inhibitors as described herein may be used as anti-metastasis inhibitors for the prevention of cancer spread. Alone or in combination with chemotherapy, radiation therapy or both, these inhibitors may improve patient survival by limiting further dissemination of disease.

Collectively, the findings presented herein suggest that inhibitors of SLK, for example, but not limited to by nm23 or CLIM1/2 peptide mimics or small molecules that have affinity for SLK may be beneficial in treatment of cancer including invasive cancers. Further, inhibitors of SLK may be beneficial in preventing cancer and tumor cells from metastasizing. Accordingly, the present invention contemplates protein variants of naturally occurring nm23, CLIM1/2, or fragments thereof that interfere with SLK activity. Furthermore, the present invention contemplates nucleic acids that encode said variants of nm23, CLIM1/2 or fragments thereof.

### REFERENCES

1. Wagner, S., Flood, T. A., O'Reilly, P., Hume, K., and Sabourin, L. A. (2002) J Biol Chem 277, 37685-37692
2. Evans, T., Rosenthal, E. T., Youngblom, J., Distel, D., and Hunt, T. (1983) Cell 33, 389-396.
3. Pines, J. (1995) Biochem J 308 (Pt 3), 697-711
4. Smits, V. A., and Medema, R. H. (2001) Biochim Biophys Acta 1519, 1-12
5. Sherr, C. J., and Roberts, J. M. (1999) Genes Dev 13, 1501-1512
6. Piaggio, G., Farina, A., Perrotti, D., Manni, I., Fuschi, P., Sacchi, A., and Gaetano, C. (1995) Exp Cell Res 216, 396-402
7. Pines, J., and Hunter, T. (1989) Cell 58, 833-846
8. Nurse, P. (1990) Nature 344, 503-508
9. Liu, F., Stanton, J. J., Wu, Z., and Piwnica-Worms, H. (1997) Mol Cell Biol 17, 571-583
10. Booher, R. N., Holman, P. S., and Fattaey, A. (1997) J Biol Chem 272, 22300-22306
11. Parker, L. L., and Piwnica-Worms, H. (1992) Science 257, 1955-1957
12. Dunphy, W. G., and Kumagai, A. (1991) Cell 67, 189-196
13. Millar, J. B., McGowan, C. H., Lenaers, G., Jones, R., and Russell, P. (1991) Embo J 10, 4301-4309
14. Lee, M. S., Ogg, S., Xu, M., Parker, L. L., Donoghue, D. J., Maller, J. L., and Piwnica-Worms, H. (1992) Mol Biol Cell 3, 73-84
15. Hoffmann, I., Clarke, P. R., Marcote, M. J., Karsenti, E., and Draetta, G. (1993) Embo J 12, 53-63
16. Izumi, T., and Maller, J. L. (1993) Mol Biol Cell 4, 1337-1350
17. Pines, J., and Hunter, T. (1991) J Cell Biol 115, 1-17
18. Roth, S. Y., Collini, M. P., Draetta, G., Beach, D., and Allis, C. D. (1991) Embo J 10, 2069-2075
19. Blangy, A., Lane, H. A., d'Herin, P., Harper, M., Kress, M., and Nigg, E. A. (1995) Cell 83, 1159-1169
20. Yamashiro, S., Yamakita, Y., Ishikawa, R., and Matsumura, F. (1990) Nature 344, 675-678
21. Kumagai, A., and Dunphy, W. G. (1996) Science 273, 1377-1380
22. Qian, Y. W., Erikson, E., and Maller, J. L. (1998) Science 282, 1701-1704
23. Liu, X., and Erikson, R. L. (2002) Proc Natl Acad Sci USA 99, 8672-8676
24. Bradbury, E. M., Inglis, R. J., Matthews, H. R., and Sarner, N. (1973) Eur J Biochem 33, 131-139
25. Allis, C. D., and Gorovsky, M. A. (1981) Biochemistry 20, 3828-3833
26. Hendzel, M. J., Wei, Y., Mancini, M. A., Van Hooser, A., Ranalli, T., Brinkley, B. R., Bazett-Jones, D. P., and Allis, C. D. (1997) Chromosoma 106, 348-360
27. Shah, J. V., and Cleveland, D. W. (2000) Cell 103, 997-1000
28. Peters, J. M. (2002) Mol Cell 9, 931-943
29. Scholey, J. M., Brust-Mascher, I., and Mogilner, A. (2003) Nature 422, 746-752
30. Sabourin, L. A., and Rudnicki, M. A. (1999) Oncogene 18, 7566-7575
31. Sabourin, L. A., Tamai, K., Seale, P., Wagner, J., and Rudnicki, M. A. (2000) Mol Cell Biol 20, 684-696
32. Zhang, Y. H., Hume, K., Cadonic, R., Thompson, C., Hakim, A., Staines, W., and Sabourin, L. A. (2002) Brain Res Dev Brain Res 139, 205-215
33. Ellinger-Ziegelbauer, H., Karasuyama, H., Yamada, E., Tsujikawa, K., Todokoro, K., and Nishida, E. (2000) Genes Cells 5, 491-498
34. Rusan, N. M., Fagerstrom, C. J., Yvon, A.-M. C., and Wadsworth, P. (2001) Mol. Biol. Cell 12, 971-980
35. Vicogne, J., Cailliau, K., Tulasne, D., Browaeys, E., Yan, Y. T., Fafeur, V., Vilain, J. P., Legrand, D., Trolet, J., and Dissous, C. (2004) J. Biol. Chem. 279, 37407-37414
36. Geley, S., Kramer, E., Gieffers, C., Gannon, J., Peters, J. M., and Hunt, T. (2001) J Cell Biol 153, 137-148
37. den Elzen, N., and Pines, J. (2001) J. Cell Biol. 153, 121-136
38. Pines, J., and Rieder, C. L. (2001) Nat Cell Biol 3, E3-6
39. Schmitt, A., and Nebreda, A. R. (2002) J Cell Sci 115, 2457-2459
40. Walter, S. A., Cutler, R. E., Jr., Martinez, R., Gishizky, M., and Hill, R. J. (2003) J Biol Chem 278, 18221-18228
41. Mustapha Bahassi, E. L., Hennigan, R. F., Myer, D. L., and Stambrook, P. J. (2004) Oncogene 21
42. WO 00/49139
   started adding more:
43. Storbeck, C. J. et al. Ste20-like kinase SLK displays myofiber type specificity and is involved in C2C12 myoblast differentiation. Muscle Nerve 29, 553-64 (2004).
44. O'Reilly, P. G. et al. The Ste20-like kinase SLK is required for cell cycle progression through G2. J Biol Chem 280, 42383-90 (2005).
45. Nishita, M. et al. Spatial and temporal regulation of cofilin activity by LIM kinase and Slingshot is critical for directional cell migration. J Cell Biol 171, 349-59 (2005).
46. Brown, M. C. & Turner, C. E. Roles for the tubulin- and PTP-PEST-binding paxillin LIM domains in cell adhesion and motility. Int J Biochem Cell Biol 34, 855-63. (2002).
47. Agulnick, A. D. et al. Interactions of the LIM-domain-binding factor Ldb1 with LIM homeodomain proteins. Nature 384, 270-2. (1996).
48. Bach, I., Carriere, C., Ostendorff, H. P., Andersen, B. & Rosenfeld, M. G. A family of LIM domain-associated cofactors confer transcriptional synergism between LIM and Otx homeodomain proteins. Genes Dev 11, 1370-80 (1997).
49. Jurata, L. W., Kenny, D. A. & Gill, G. N. Nuclear LIM interactor, a rhombotin and LIM homeodomain interacting protein, is expressed early in neuronal development. Proc Natl Acad Sci USA 93, 11693-8 (1996).
50. Mukhopadhyay, M. et al. Functional ablation of the mouse Ldb1 gene results in severe patterning defects during gastrulation. Development 130, 495-505 (2003).
51. Thaler, J. P., Lee, S. K., Jurata, L. W., Gill, G. N. & Pfaff, S. L. LIM factor Lhx3 contributes to the specification of motor neuron and interneuron identity through cell-type-specific protein-protein interactions. Cell 110, 237-49 (2002).
52. Etienne-Manneville, S. & Hall, A. Integrin-mediated activation of Cdc42 controls cell polarity in migrating astrocytes through PKCzeta. Cell 106, 489-98 (2001).
53. Ezratty, E. J., Partridge, M. A. & Gundersen, G. G. Microtubule-induced focal adhesion disassembly is mediated by dynamin and focal adhesion kinase. Nat Cell Biol 7, 581-90 (2005).
54. Matthews, J. M. & Visvader, J. E. LIM-domain-binding protein 1: a multifunctional cofactor that interacts with diverse proteins. EMBO Rep 4, 1132-7 (2003).
55. Kaverina, I., Rottner, K. & Small, J. V. Targeting, capture, and stabilization of microtubules at early focal adhesions. J Cell Biol 142, 181-90. (1998).
56. Kaverina, I., Krylyshkina, O. & Small, J. V. Microtubule targeting of substrate contacts promotes their relaxation and dissociation. J Cell Biol 146, 1033-44. (1999).
57. van Meyel, D. J. et al. Chip is an essential cofactor for apterous in the regulation of axon guidance in Drosophila. Development 127, 1823-31 (2000).
58. Lei, M. et al. Structure of PAK1 in an autoinhibited conformation reveals a multistage activation switch. Cell 102, 387-97 (2000).
59. Rudel, T. & Bokoch, G. M. Membrane and morphological changes in apoptotic cells regulated by caspase-mediated activation of PAK2. Science 276, 1571-4 (1997).
60. Zhang, Y.-H. et al. Expression of the Ste20-like kinase SLK during embryonic development and in the murine adult central nervous system. Brain Res Dev Brain Res 139, 205-15 (2002).

All citations are herein incorporated by reference.

The present invention has been described with regard to preferred embodiments. However, it will be obvious to persons skilled in the art that a number of variations and modifications can be made without departing from the scope of the invention as described herein.

### SEQUENCES

| SEQ ID NO:1 | |
|---|---|
| 1 msffnfrkif klgsekkkkq yehvkrdlnp eefweiigel gdgafgkvyk aqnketnvla | |
| 61 aakvidtkse eeledymvei dilascdhpn ivklldafyy ennlwilief caggavdavm | |
| 121 lelerpltes qiqvvckqtl ealnylhdnk iihrdlkagn ilftldgdik ladfgvsakn | |
| 181 trtiqrrdsf igtpywmape vvmcetskdr pydykadvws lgitliemae iepphhelnp | |
| 241 mrvllkiaks epptlaqpsk wssnfkdflr kcleknvdar wttsqllqhp fvtvdsnkpv | |
| 301 reliaeakae vteevedgke edeeeeaena lpipankras sdlsiassee dklsqnacil | |
| 361 esvserteqs tsedkfsnki lnekpttdgp ekavdehasd vnletgaeln dqtvgiheng | |
| 421 rekkrpklen lpdtqdqqtv dvnsvseene nnrvtletnt dclkpeedrn kenqetlesk | |
| 481 liqseeindt hiqtmdlvsq etgekeadfq avdnevgltk eetqeklgkd gtaqkvitsd | |
| 541 rssevgtdea lddtqkaael skaaqsgegd ealaptqtla ekptegpeag gaeeeppgge | |
| 601 rvedkqpeqq pavceaegql tstsettrat leqpetdeve qvsesnsiee lerlvvtgae | |
| 661 aralgsegea aatevdlerk enaqkvpvka esqapaasqp sephpvlips ininsetten | |
| 721 keemgaipkp etilppepeh ekgndtdsgt gstvenssgd lnlsissfls kakdsgsvsl | |
| 781 qetrrqkktl kktrkfivdg vevsvttski vtdsdsktee lrflrrqelr elrllqkeeq | |
| 841 raqqqlngkl qqqreqifrr feqemlskkr qydqeienle kqqkqtierl eqehtnrlrd | |
| 901 eakrikgeqe kelskfqnvl knrkkeeqef vqkqqqeldg slkkiiqqqk aelanierec | |
| 961 lnnkqqlmra reaaiwelee rhlqekhqll kqqlkdqyfm qrhqllkrhe keteqmqryn | |
| 1021 qrlieelknr qtqerarlpk iqrseaktrm amfkkslrin statpdqdre kikqfaaqee | |
| 1081 krqknermaq hqkhesqmrd lqlqceanvr elhqlqnekc hllvehetqk lkeldeehsq | |
| 1141 elkewreklr prkktieeef arklqeqevf fkmtgesecl npsaqsrisk fypiptlhst | |
| 1201 gs | |
| SEQ ID NO:2 | |
| 1 msffnfrkif klgsekkkkq yehvkrdlnp eefweiigel gdgafgkvyk aqnketnvla | |
| 61 aarvidtkse eeledymvei dilascdhpn ivklldafyy enniwilief caggavdavm | |
| 121 lelerpltes qiqvvckqtl ealnylhdnk iihrdlkagn ilftldgdik ladfgvsakn | |
| 181 trtiqrrdsf igtpywmape vvmcetskdr pydykadvws lgitliemae iepphhelnp | |
| 241 mrvllkiaks epptlaqpsk wssnfkdflr kcleknvdar wttsqllqhp fvtvdsnkpv | |
| 301 reliaeakae vteevedgke edeeeeaena lpipankras sdlsiassee dklsqnacil | |
| 361 esvserteqs tsedkfsnki lnekpttdgp ekavdehasd vnletgaeln dqtvgiheng | |
| 421 rekkrpklen lpdtqdqqtv dvnsvseene nnrvtletnt dclkpeedrn kenqetlesk | |
| 481 liqseeindt hiqtmdlvsq etgekeadfq avdnevgltk eetqeklgkd gtaqkvitsd | |
| 541 rssevgtdea lddtqkaael skaaqsgegd ealaptqtla ekptegpeag gaeeeppgge | |
| 601 rvedkqpeqq pavceaegql tstsettrat leqpetdeve qvsesnsiee lerlvvtgae | |
| 661 aralgsegea aatevdlerk enaqkvpvka esqapaasqp sephpvlips ininsetten | |
| 721 keemgalpkp etilppepeh ekgndtdsgt gstvenssgd lnlsissfls kakdsgsvsl | |
| 781 qetrrqkktl kktrkfivdg vevsvttski vtdsdsktee lrflrrqelr elrllqkeeq | |
| 841 raqqqlngki qqqreqifrr feqemlskkr qydqeienle kqqkqtierl eqehtnrlrd | |
| 901 eakrikgeqe kelskfqnvl knrkkeeqef vqkqqqeldg slkkiiqqqk aelanierec | |
| 961 lnnkqqlmra reaaiwelee rhlqekhqll kqqlkdqyfm qrhqlikrhe keteqmqryn | |
| 1021 qrlieelknr qtqerarlpk iqrseaktrm amfkkslrin statpdqdre kikqfaaqee | |
| 1081 krqknermaq hqkhesqmrd lqlqceanvr elhqlqnekc hllvehetqk lkeldeehsq | |
| 1141 elkewreklr prkktleeef arklqeqevf fkmtgesecl npsaqsrisk fypiptlhst | |
| 1201 gs | |
| GGTTGAGATTGACATATTA | (SEQ ID NO:3) |
| GATAATTTATGGATGTGAC, | (SEQ ID NO:4) |
| UAGCGACUAAACACAUCAAUU, | (SEQ ID NO:5) |
| GGUUGAGAUUGACAUAUUA | (SEQ ID NO:6) |
| ACAAGCAGCACGUCCAAUAUU | (SEQ ID NO:7) |
| GAACUUAUGUCCCGCCACAUU | (SEQ ID NO:8) |
| ggggggatccagctgcaaacctgagtctgtcc | (SEQ ID NO:9) |
| gggggaattcacgggcctattgacagtggattct | (SEQ ID NO:10) |

The embodiments of the invention in which an exclusive property of privilege is claimed are defined as follows:

1. A method of inhibiting the proliferation, motility or both the proliferation and motility of a cell in a subject comprising administering an SLK inhibitor to said subject.

2. The method of claim 1, wherein the cell is a cancer or tumor cell.

3. The method as defined in claim 1 wherein the subject is a human subject.

4. The method of claim 1, wherein said SLK inhibitor is provided in a virus.

5. The method of claim 4, wherein the virus is an adenovirus, lentivirus or a retrovirus.

6. The method of claim 1, wherein said SLK inhibitor comprises a catalytically inactive SLK or a nucleic acid encoding a catalytically inactive SLK kinase.

7. The method of claim 1, wherein said SLK inhibitor comprises a fragment of SLK.

8. The method of claim 1 wherein said SLK inhibitor is an antisense nucleic acid or a short interfering RNA (siRNA).

9. The method of claim 1, wherein said SLK inhibitor is an antibody or fragment thereof which is capable of binding SLK.

10. The method of claim 1, wherein said SLK inhibitor is a binding partner of SLK, a variant of a naturally occurring binding partner, or a fragment of a binding partner of SLK.

11. A cell comprising an SLK inhibitor.

12. The cell according to claim 11, wherein said cell is a non-cultured cell.

13. The cell of claim 11, wherein said cell is a cancer or a tumour cell.

14. The cell of claim 11, wherein said cell is a HER2+ carcinoma or sarcinoma.

15. The cell of claim 11, wherein the SLK inhibitor is a heterologous SLK inhibitor which is not found within the cell in nature.

16. An in vitro method of inhibiting the proliferation, motility or both the proliferation and motility of a cell comprising administering an SLK inhibitor to said cell.

17. The method of claim 16, wherein the cell is a cancer or tumor cell.

18. The method as defined in claim 16 wherein the cell is a human cell.

19. The method of claim 16, wherein said SLK inhibitor is encoded by a virus.

20. The method of claim 19, wherein the virus is an adenovirus, lentivirus or a retrovirus.

21. The method of claim 16, wherein said SLK inhibitor comprises a catalytically inactive SLK or a nucleic acid encoding a catalytically inactive SLK kinase.

22. The method of claim 16, wherein said SLK inhibitor comprises a fragment of SLK.

23. The method of claim 16 wherein said SLK inhibitor is an antisense nucleic acid or a short interfering RNA (siRNA).

24. The method of claim 16, wherein said SLK inhibitor is an antibody or fragment thereof which is capable of binding SLK.

25. The method of claim 16, wherein said SLK inhibitor is a binding partner of SLK, a variant of a naturally occurring binding partner, or a fragment of a binding partner of SLK.

26. A method of screening a compound to determine if said compound is effective as an anticancer agent, the method comprising,
a) selecting a first group of cells and a second group of cells,
b) treating the first group of cells with the compound;
c) measuring SLK kinase activity, proliferation, motility, migration or any combination thereof of said first group of cells relative to the second group of cells, wherein a measurable decrease in SLK kinase activity, proliferation, motility, migration or any combination thereof in said first group of cells relative to said second group of cells indicates that said compound is effective as an anticancer agent.

27. The method of claim 26, wherein said first group of cells are cancer or tumor cells and said second group of cells are non-cancerous normal cells of the same cell type.

28. The method of claim 27, wherein the non-cancerous normal cells and the cancer or tumor cells are from the same cell line.

29. The method of claim 28, wherein the non-cancerous normal cells and the cancer or tumor cells are obtained from a subject.

30. The method of claim 26, wherein the second group of cells exhibit contact inhibition of growth whereas the first group of cells do not.

31. The method of claim 27, wherein said second group of cells is also treated with the compound in a manner that is substantially similar to the first group of cells.
